(19) United States
(12) Patent Application Publication
(10) Pub. No.: US 2009/0054414 A1
(43) Pub. Date: Feb. 26, 2009

(54) Rufinamide for the Treatment of Post-Traumatic Stress Disorder

(75) Inventors: Tom Woiwode, South San Francisco, CA (US); Mark Moran, South San Francisco, CA (US)

Correspondence Address: FENWICK & WEST LLP, SILICON VALLEY CENTER, 801 CALIFORNIA STREET, MOUNTAIN VIEW, CA, 94041, US

(73) Assignee: SYNOSIA THERAPEUTICS, South San Francisco, CA (US)

(21) Appl. No.: 12/178,502

(22) Filed: Jul. 23, 2008

Related U.S. Application Data
(60) Provisional application No. 60/935,032, filed on Jul. 23, 2007.

(51) Int. Cl. A61K 31/5513; A61K 31/4196

(52) U.S. Cl. 514/221; 514/359

(57) ABSTRACT

Provided are methods of treating post-traumatic stress disorder with rufinamide. Also provided are methods of treating kindling and improving resilience with rufinamide. Also provided are methods of diagnosing post-traumatic stress disorder in a patient by administering to the patient a therapeutically effective amount of rufinamide and assessing at least one of sign, symptom, or symptom cluster of post-traumatic stress disorder; and diagnosing post-traumatic stress disorder in the patient if the rufinamide reduces at least one of sign, symptom, and symptom cluster of post-traumatic stress disorder.

### CROSS-REFERENCE TO RELATED APPLICATIONS

This application claims the benefit of and priority under 35 U.S.C. § 119(e) to U.S. Provisional Patent Application Ser. No. 60/935,032, “RUFINAMIDE FOR THE TREATMENT OF POST-TRAUMATIC STRESS DISORDER” filed Jul. 23, 2007, which is incorporated herein by reference in its entirety.

### FIELD OF INVENTION

This relates generally to methods for treating post-traumatic stress disorder and more particularly methods of treating post-traumatic stress disorder with rufinamide. Also provided are methods of treating kindling and improving resilience with rufinamide. Also provided are methods of diagnosing post-traumatic stress disorder in a patient by administering to the patient a therapeutically effective amount of rufinamide and assessing at least one of sign, symptom, or symptom cluster of post-traumatic stress disorder; and diagnosing post-traumatic stress disorder in the patient if the rufinamide reduces at least one of sign, symptom, and symptom cluster of post-traumatic stress disorder, among other things.

### BACKGROUND OF THE INVENTION

Anxiety disorders are the most commonly occurring disorders of the psychiatric illnesses with an immense economic burden. In addition to generalized anxiety disorder, they encompass post-traumatic stress disorder, panic disorder, obsessive compulsive disorder and social as well as other phobias.

Post-traumatic stress disorder can be severe and chronic, with some studies suggesting a lifetime prevalence of 1.3% to 7.8% in the general population. Post-traumatic stress disorder typically follows a psychologically distressing traumatic event. These events may include military combat, terrorist incidents, physical assault, sexual assault, motor vehicle accidents, and natural disasters, for example. The response to the event can involve intense fear, helplessness, or horror. Most people recover from the traumatic event with time and return to normal life. In contrast, in post-traumatic stress disorder victims, symptoms persist and may worsen with time, preventing a return to normal life.

Psychotherapy is currently the backbone of post-traumatic disorder treatment. Methods include cognitive-behavioral therapy, exposure therapy, and eye movement desensitization and reprocessing. Medication can enhance the effectiveness of psychotherapy. Selective serotonin reuptake inhibitors (SSRIs), such as sertraline (Zoloft®) and paroxetine (Paxil®), are the only medications approved for treating PTSD by the Food and Drug Administration. Many unwanted side effects and characteristics are associated with SSRI usage. These include concerns about drug interactions, gastrointestinal side effects, sexual side effects, suicidal ideation, acute anxiogenic effects, and slow onset of action. Some tricyclic antidepressants (TCAs) and monamine oxidase inhibitors (MAOIs) appear to have some efficacy but patient tolerance is low due to the high incidence of side effects. MAOIs have dietary restriction requirements and are linked to hypertensive events. TCAs have anticholinergic and cardiovascular side effects. Lamotrigine, a sodium channel blocker, has had some efficacy in treating post-traumatic stress disorder in a small scale placebo controlled study. Difficulty in the use of lamotrigine due the to necessity for titration and the risk of developing Steven Johnson Syndrome, a life threatening rash, render it a poor candidate for therapeutic use.

There is a need for the development of treatments for post-traumatic stress disorder that are safe and effective.

Rufinamide (1-(2,6-difluorobenzyl)-1H-1,2,3-triazole-4-carboxamide) is a sodium channel modulator and effective anticonvulsant drug. Sodium channels participate in neurotransmission through generation of action potentials. Rufinamide is a fluorinated triazole. Fluorinated triazoles may be used to treat affective disorders. See U.S. Pat. No. 6,156,775.

### SUMMARY OF THE INVENTION

Provided are methods of treating a patient diagnosed with post-traumatic stress disorder. The methods include administering to the patient a therapeutically effective amount of rufinamide (1-(2,6-difluorobenzyl)-1H-1,2,3-triazole-4-carboxamide). In certain embodiments the rufinamide reduces kindling in the patient.

Also provided are methods of treating kindling in a patient. The methods include administering to the patient a therapeutically effective amount of rufinamide. In certain embodiments the rufinamide reduces the incidence of at least one of epilepsy and post-traumatic stress disorder in the patient.

Also provided are methods of treating post-traumatic stress disorder in a patient. The methods include diagnosing the patient with post-traumatic stress disorder; administering to the patient a therapeutically effective amount of rufinamide; assessing at least one of sign, symptom, and symptom cluster of post-traumatic stress disorder; and determining that the post-traumatic stress disorder is improved if the rufinamide reduces at least one of sign, symptom, and symptom cluster of post-traumatic stress disorder.

Also provided are methods of improving resilience in a patient. The methods include administering a therapeutically effective amount of rufinamide.

Also provided are methods of diagnosing post-traumatic stress disorder in a patient. The methods include administering to the patient a therapeutically effective amount of rufinamide and assessing at least one of sign, symptom, or symptom cluster of post-traumatic stress disorder; and diagnosing post-traumatic stress disorder in the patient if the rufinamide reduces at least one of sign, symptom, and symptom cluster of post-traumatic stress disorder. In certain embodiments the patient is a child, adolescent, or adult.

### DESCRIPTION OF DRAWINGS

FIG. 1 Shows Chemical Structure of Rufinamide

FIG. 2 Shows Effects of Oral Administration of Rufinamide on Social Exploration in Rats

FIG. 3 Shows the Rodent Ethogram Used on the Social Withdrawal of Intruder Mice Test

FIG. 4 Shows Ethological Profile of Rufinamide on Male Intruder Mice

FIG. 5 Shows Ethological Profile of Carbamazepine on Male Intruder Mice

FIG. 6 Shows Ethological Profile of Rufinamide on Male Intruder Mice

FIG. 7 Shows Ethological Profile of Carbamazepine on Male Intruder Mice

FIG. 8 Shows Effects of Rufinamide on the Rectal Temperature of Male of1 Mice

FIG. 9 Shows Effects of Carbamazepine on the Rectal Temperature of Male of1 Mice

FIG. 10 Shows the Ethogram used in the Half Enclosed Platform test.

FIG. 11 Shows the Effects of Rufinamide on the Behaviour of Male of1 Mice Exploring a Half-Enclosed Platform

FIG. 12 Shows the Effects of Rufinamide on the Behaviour of Male of1 Mice Exploring a Half-Enclosed Platform

FIG. 13 Shows the Effects of Carbamazepine on the Behaviour of Male of1 Mice Exploring a Half-Enclosed Platform

FIG. 14 Shows the Effects of Carbamazepine on the Behaviour of Male of1 Mice Exploring a Half-Enclosed Platform

FIG. 15 Shows Effects of Rufinamide on the Rectal Temperature of Male of1 Mice

FIG. 16 Shows Effects of Carbamazepine on the Rectal Temperature of Male of1 Mice

FIG. 17 Shows the Effects of Rufinamide (3-100 mg/kg) on the stress-induced hyperthermia (SIH) response in mice.

FIG. 18 Shows the Effects of Rufinamide (3-100 mg/kg) on basal temperature and on temperature following stress in mice

FIG. 19 Shows the Effects of Rufinamide (0.3-300 mg/kg) on the stress-induced hyperthermia (SIH) response in mice

FIG. 20 Shows the Effects of Rufinamide (3-100 mg/kg) on basal temperature and on temperature following stress in mice

FIG. 21 Shows the Effects of Rufinamide (25-200 mg/kg) on the stress-induced hyperthermia (SIH) response in mice

FIG. 22 Shows the Effects of Rufinamide (3-100 mg/kg) on basal temperature and on temperature following stress in mice

FIG. 23 Shows the Total Data-Effects of Rufinamide (0.0-300 mg/kg) on the stress-induced hyperthermia (SIH) response in mice

FIG. 24 Shows the Effects of Rufinamide (0.0-300 mg/kg) on basal temperature and on temperature following stress in mice

FIG. 25 Shows the Effects of Rufinamide and Clobazam in the Vogel Conflict Test in the Rat

FIG. 26 The Effects of Rufinamide and Clobazam in the Vogel Conflict Test in the rat.

### DETAILED DESCRIPTION

As used herein, the following words and phrases are generally intended to have the meanings as set forth below, except to the extent that the context in which they are used indicates otherwise.

As used herein “rufinamide (1-(2,6-difluorobenzyl)-1H-1,2,3-triazole-4-carboxamide)” includes (1-(2,6-difluorobenzyl)-1H-1,2,3-triazole-4-carboxamide), as well as pharmaceutically acceptable salts thereof.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)n—COOH where n is 0-4, and like salts.

In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, the term “treating” refers to any manner in which at least one sign, symptom, or symptom cluster of a disease or disorder is beneficially altered so as to prevent or delay the onset, reduce the incidence or frequency, reduce the severity or intensity, retard the progression, prevent relapse, or ameliorate the symptoms or associated symptoms of the disease or disorder. For example, in post-traumatic stress disorder, treating the disorder can, in certain embodiments, cause a reduction in at least one of the frequency and intensity of at least one of a sign, symptom, and symptom cluster of post-traumatic stress disorder.

As used herein the phrase “diagnosed with post-traumatic stress disorder (PTSD)” refers to having a sign, symptom, or symptom cluster indicative of post-traumatic stress disorder, a psychiatric disorder triggered by a traumatic event. Non-limiting examples of such traumatic events include military combat, terrorist incidents, physical assault, sexual assault, motor vehicle accidents, and natural disasters.

The Diagnostic and Statistical Manual of Mental Disorders-IV-Text revised (DSM-IV-TR), a handbook for mental health professionals that lists categories of mental disorders and the criteria, classifies post-traumatic stress disorder as an anxiety disorder. According to the DSM-IV-TR, a PTSD diagnosis can be made if:

1. the patient experienced, witnessed, or was confronted with an event or events that involved actual or threatened death or serious injury, or a threat to the physical integrity of self or others and the response involved intense fear, helplessness, or horror;

2. as a consequence of the traumatic event, the patient experiences at least 1 re-experiencing/intrusion symptom, 3 avoidance/numbing symptoms, and 2 hyperarousal symptoms, and the duration of the symptoms is for more than 1 month; and

3. the symptoms cause clinically significant distress or impairment in social, occupational, or other important areas of functioning.

In certain embodiments, if the patient's disorder fulfills DSM-IV-TR criteria, the patient is diagnosed with post-traumatic stress disorder. In certain embodiments, if the patient has at least one sign, symptom, or symptom cluster of post-traumatic stress disorder, the patient is diagnosed with post-traumatic stress disorder. In certain embodiments, a scale is used to measure a sign, symptom, or symptom cluster of post-traumatic stress disorder, and post-traumatic stress disorder is diagnosed on the basis of the measurement using that scale. In certain embodiments, a “score” on a scale is used to diagnose or assess a sign, symptom, or symptom cluster of post-traumatic stress disorder. In certain embodiments, a “score” can measure at least one of the frequency, intensity, or severity of a sign, symptom, or symptom cluster of post-traumatic stress disorder.

As used herein, the term “scale” refers to a method to measure at least one sign, symptom, or symptom cluster of post-traumatic stress disorder in a patient. In certain embodiments, a scale may be an interview or a questionnaire. Non-limiting examples of scales are Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

As used herein, the terms “sign” and “signs” refer to objective findings of a disorder. In certain embodiments, a sign can be a physiological manifestation or reaction of a disorder. In certain embodiments, a sign may refer to heart rate and rhythm, body temperature, pattern and rate of respiration, blood pressure. In certain embodiments, signs can be associated with symptoms. In certain embodiments, signs can be indicative of symptoms.

As used herein, the term “symptom” and “symptoms” refer to subjective indications that characterize a disorder. Symptoms of post-traumatic stress disorder may refer to, for example, but not limited to recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response. In certain embodiments, potentially threatening stimuli can cause hyperarousal or anxiety. In certain embodiments, the physiological reactivity manifests in at least one of abnormal respiration, abnormal cardiac rate of rhythm, abnormal blood pressure, abnormal function of a special sense, and abnormal function of sensory organ. In certain embodiments, restricted range of effect characterized by diminished or restricted range or intensity of feelings or display of feelings can occur and s sense of a foreshortened future can manifest in thinking that one will not have a career, marriage, children, or a normal life span. In certain embodiments, children and adolescents may have symptoms of post-traumatic stress disorder such as, for example and without limitation, disorganized or agitated behavior, repetitive play that expresses aspects of the trauma, frightening dreams which lack recognizable content, and trauma-specific reenactment.

As used herein, the term “symptom cluster” refers to a set of signs, symptoms, or a set of signs and symptoms, that are grouped together because of their relationship to each other or their simultaneous occurrence. For example, in certain embodiments post-traumatic stress disorder is characterized by three symptom clusters: re-experiencing/intrusion, avoidance/numbing, and hyperarousal.

As used herein, the term “re-experiencing/intrusion” refers to at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders. In certain embodiments, the physiological reactivity manifests in at least one of abnormal respiration, abnormal cardiac rate of rhythm, abnormal blood pressure, abnormal function of a special sense, and abnormal function of sensory organ.

As used herein, the term “avoidance/numbing” refers to at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, and sense of a foreshortened future. Restricted range of effect characterized by diminished or restricted range or intensity of feelings or display of feelings can occur. A sense of a foreshortened future can manifest in thinking that one will not have a career, marriage, children, or a normal life span. Avoidance/numbing can also manifest in social anxiety and anxiety with unfamiliar surroundings.

As used herein, the term “hyperarousal” refers to at least one of difficulty falling or staying asleep, irritability or outbursts of anger, difficulty concentrating, hypervigilance, and exaggerated startle response. Potentially threatening stimuli can cause hyperarousal or anxiety.

As used herein, the term “significantly” refers to a set of observations or occurrences that are too closely correlated to be attributed to chance. For example, in certain embodiments, “significantly changes”, “significantly reduces”, and “significantly increases” refers to alterations or effects that are not likely to be attributed to chance. In certain embodiments, statistical methods can be used to determine whether an observation can be referred to as “significantly” changed, reduced, increased, or altered. In certain embodiments, a “score” that assesses post-traumatic stress disorder can be significantly changed, for example, by treatment for post-traumatic stress disorder.

Patients diagnosed with post-traumatic stress disorder may feel “on guard”, uneasy, and intensely anxious. Depression, anxiety, panic attacks, and bipolar disorder are often associated with post-traumatic stress disorder. Alcohol and drug abuse are also common. In certain embodiments, disorders cormorbid with post-traumatic stress disorder can include for example but without limitation depression, alcohol abuse, and drug abuse.

As used herein, the term “Clinician-Administered PTSD Scale (CAPS)” refers to a measure for diagnosing and assessing post-traumatic stress syndrome. The CAPS is a 30-item structured interview that corresponds to the DSM-IV criteria for PTSD. Different versions of this measure have been developed.

As used herein, the term “Clinician-Administered PTSD Scale-Part 1 (CAPS-1)” is a version of CAPS that assesses current and lifetime PTSD and is also known as CAPS-DX (for diagnosis).

As used herein, the term “Clinician-Administered PTSD Scale-Part 2 (CAPS-2)” refers to a version of CAPS used to assess one week symptom status in patients with post-traumatic stress disorder and also refers to a CAPS-SX (for symptom).

As used herein, the term “Clinician-Administered PTSD Scale for children and adolescents (CAPS-CA)” refers to a version of CAPS developed for children and adolescents.

As used herein, the term “Impact of Event Scale (IES)” refers to a scale developed by Mardi Horowitz, Nancy Wilner, and William Alvarez to measure subjective stress related to a specific event. It is a self-reported assessment and can be used to make measurements over time to monitor a patient's status.

As used herein, the term “Impact of Event Scale-Revised (IES-R)” refers to the revision of the IES developed by Daniel S. Weiss and Charles Marmar to assess the hyperarousal symptom cluster of PTSD.

As used herein, the term “Clinical Global Impression Scale (CGI)” refers to a scale for making psychiatric assessments. Patients are interviewed and the CGI is used to measure the severity of illness (CGI-S), global improvement (CGI-I), and efficacy index.

As used herein, the term “Clinical Global Impression Severity of Illness (CGI-S)” refers to an assessment of the patient's current symptoms. Generally, it is rated on a seven-point scale, ranging from a score of 1 (normal) to 7 (extremely ill). The severity of the patient's illness is compared to the severity of other patients' illness. For example, the CGI-S score can be used to measure a patient's condition after treatment with rufinamide, and the scores before and after treatment may be compared.

As used herein, the term “Clinical Global Impression Improvement (CGI-I)” refers to a comparison of a patient's current condition to his baseline condition. Generally, it is rated on a seven-point scale ranging from 1 (very much improved) to 7 (very much worse). The CGI-I score can be used to measure, for example, improvement of post-traumatic stress disorder in response to rufinamide treatment.

As used herein, the term “efficacy index” refers to a score taken on CGI and compares the patient's baseline condition with a ratio of current therapeutic benefit to severity of side effects. Generally, it is rated on a four-point scale ranging from 1 (none) to 4 (outweighs therapeutic effect). In assessing post-traumatic stress disorder, the efficacy index could, for example, assess the risk-benefit of treating with a therapy such as rufinamide.

As used herein, the term “Duke Global Rating for PTSD scale (DGRP)” refers to a scale that measures severity and improvement for each of the three PTSD symptom clusters: re-experiencing/intrusion, avoidance/numbing, and hyperarousal as well as overall PTSD severity.

As used herein, the term “Duke Global Rating for PTSD scale-Improvement (DGRP-I)” refers to a scale used to distinguish responders (DGRP-I of 1 (very much improved) and 2 (much improved)) from nonresponders (DGRP-I>2) of in response to a treatment, for example, rufinamide, for post-traumatic stress disorder.

As used herein, the term “Hamilton Anxiety Scale (HAM-A)” refers to a scale developed by Max Hamilton in 1959 to diagnose and quantify symptoms of anxiety and post-traumatic stress disorder. It consists of 14 items, each defined by a series of symptoms. No standardized probe questions to elicit information from patients or behaviorally specific guidelines were developed for determining item scoring. Each item is rated on a point scale, ranging from 0 (not present) to 4 (severe). Items include assessing anxious mood, fears, intellectual effects, somatic complaints, e.g. on musculature, cardiovascular symptoms, tension, insomnia, depressed mood, somatic sensory complaints, respiratory symptoms, gastrointestinal symptoms, autonomic symptoms, genitourinary symptoms, and behavior at the time of assessment. For example, a reduction in the HAM-A score would indicate improvement in a disorder such as post-traumatic stress disorder.

As used herein, the terms “Structured Interview for PTSD (SI-PTSD), PTSD

Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI)” refer to additional scales that diagnose, assess, measure a sign, symptom, symptom cluster of post-traumatic stress disorder, anxiety, or depression.

As used herein, the term “score” refers to a score of at least one item or parameter measured on a scale that measures at least one sign, symptom, or symptom cluster of psychiatric symptoms, anxiety, or post-traumatic stress disorder. In certain embodiments, a score measures the frequency, intensity, or severity of a sign, symptom, symptom cluster, associated symptom, or impact on daily life of post-traumatic stress disorder.

As used herein, the term “endpoint score” refers to a score on an instrument that assesses post-traumatic stress disorder taken during or after treatment.

As used herein, the term “baseline score” refers to a score on an instrument that assesses post-traumatic stress disorder prior to initiation of a treatment.

As used herein, the term “overall score” refers to a sum of the scores on an instrument that assesses post-traumatic stress disorder. In certain embodiments, an overall score is the sum of a score of at least one of symptoms, symptoms clusters, associated symptoms, impact on daily life, efficacy, and improvement.

As used herein, the term “relapse” refers to reoccurrence or worsening of at least one symptom of a disease or disorder in a patient.

As used herein the phrase “therapeutically effective amount” refers to the amount sufficient to provide a therapeutic outcome regarding at least one sign, symptom, or associated symptom of a disease, disorder, or condition. For example, the disease, disorder, or condition is PTSD.

As used herein, the term “kindling” refers to low level electrical or chemical stimulation of the brain which may act as a sensitizer and trigger neurological diseases including but not limited to epilepsy and post-traumatic stress disorder.

As used herein, the phrase “improving resilience” refers to increasing the ability of a patient to experience a traumatic event without suffering post-traumatic stress disorder or with less post-event symptomotology or disruption of normal activities of daily living. In certain embodiments, improving resilience can reduce at least one of signs, symptoms, and symptom clusters of post-traumatic stress disorder.

As used herein, the term “coadministering” refers to a dosage regimen for a first agent that overlaps with the dosage regimen of a second agent, or to simultaneous administration of the first agent and the second agent. A dosage regimen is characterized by dosage amount, frequency, and duration. Two dosage regimens overlap if between initiation of a first and initiation of a second administration of a first agent, the second agent is administered.

As used herein, the term “agent” refers to a substance including, but not limited to a chemical compound, such as a small molecule or a complex organic compound, a protein, such as an antibody or antibody fragment or a protein comprising an antibody fragment, or a genetic construct which acts at the DNA or mRNA level in an organism.

As used herein, the phrase “sodium channel activity” refers to at least one activity triggered by sodium channels. Sodium channels, also known as voltage-gated sodium channels, are integral membrane proteins that conduct sodium ions through cellular plasma membranes. There are currently nine known members of the sodium channel family: Naᵥ 1.1 to Naᵥ 1.9. By conducting sodium ions through the plasma membrane, sodium channels may depolarize the cell, propagate action potentials, and contribute to neurotransmission.

Methods of treating a patient diagnosed with post-traumatic stress disorder are provided herein. The methods include administering to the patient a therapeutically effective amount of rufinamide (1-(2,6-difluorobenzyl)-1H-1,2,3-triazole-4-carboxamide). In certain embodiments the rufinamide reduces kindling in the patient.

In certain embodiments the methods further include coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the at least one other agent is a SSRI selected from paroxetine, sertraline, citalopram, escitalopram, and fluoxetine.

In certain embodiments the at least one other agent is a SNRI selected from duloxetine, mirtazapine, and venlafaxine.

In certain embodiments the at least one other agent is a NRI selected from bupropion and atomoxetine.

In certain embodiments the at least one other agent is a dopamine β-hydroxylase inhibitor selected from nepicastat and disulfuram.

In certain embodiments the at least one other agent is the adenosine A2A receptor antagonist istradefylline.

In certain embodiments the at least one other agent is a sodium channel blocker selected from lamotrigine, carbamazepine, oxcarbazepine, and valproate.

In certain embodiments the at least one other agent is a calcium channel blocker selected from lamotrigine and carbamazepine.

In certain embodiments the at least one other agent is the central and peripheral alpha adrenergic receptor antagonist prazosin.

In certain embodiments the at least one other agent is the central alpha adrenergic agonist clonidine.

In certain embodiments the at least one other agent is the central or peripheral beta adrenergic receptor antagonist propranolol.

In certain embodiments the least one other agent is an atypical antidepressant/antipsychotic selected from olanzepine, risperidone, and quetiapine.

In certain embodiments the least one other agent is a tricyclic selected from amitriptyline, amoxapine, desipramine, doxepin, imipramine, nortriptyline, protiptyline, and trimipramine.

In certain embodiments the least one other agent is an anticonvulsant selected from lamotrigine, carbamazepine, oxcarbazepine, valproate, topiramate, and levetiracetam.

In certain embodiments the least one other agent is the glutamate antagonist topiramate.

In certain embodiments the least one other agent is a GABA agonist selected from valproate and topiramate.

In certain embodiments the least one other agent is the partial D2 agonist aripiprazole.

In certain embodiments the rufinamide reduces at least one sodium channel activity in the patient.

In certain embodiments the rufinamide reduces at least one of the frequency and intensity of at least one sign of the post-traumatic stress disorder in the patient.

In certain embodiments the rufinamide reduces at least one of the frequency and intensity of at least one symptom of the post-traumatic stress disorder in the patient.

In certain embodiments the rufinamide reduces at least one of the frequency and intensity of at least one symptom cluster of the post-traumatic stress disorder in the patient, wherein the symptom cluster is selected from re-experiencing/intrusion, avoidance/numbing, and hyperarousal.

In certain embodiments the re-experiencing/intrusion includes at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders.

In certain embodiments the physiological reactivity includes at least one of abnormal respiration, abnormal cardiac rate of rhythm, abnormal blood pressure, abnormal function of at least one special sense, and abnormal function of at least one sensory organ. In certain embodiments the at least one special sense is selected from sight, hearing, touch, smell, taste, and sense. In certain embodiments the at least one sensory organ is selected from eye, ear, skin, nose, tongue, and pharynx.

In certain embodiments of the methods the avoidance/numbing comprises at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, and anxiety associated with unfamiliar surroundings.

In certain embodiments the hyperarousal comprises at least one of difficulty falling or staying asleep, irritability or outbursts of anger, difficulty concentrating, hypervigilance, exaggerated startle response, and anxiety from potentially threatening stimuli. In certain embodiments the rufinamide does not reduce the physical ability of the patient to respond appropriately and promptly to the potentially threatening stimuli.

In certain embodiments the patient is a child or an adolescent. In certain embodiments the rufinamide reduces at least one of the frequency and intensity of at least one sign or symptom of the post-traumatic stress disorder in the patient, wherein the sign or symptom is selected from disorganized or agitated behavior, repetitive play that expresses aspects of the trauma, frightening dreams which lack recognizable content, and trauma-specific reenactment.

In certain embodiments the rufinamide reduces the incidence of at least one disorder comorbid with post-traumatic stress disorder selected from drug abuse, alcohol abuse, and depression in the patient.

In certain embodiments the rufinamide is administered to the patient once or twice a day.

In certain embodiments the therapeutically effective amount of rufinamide does not exceed 45 milligrams per day.

In certain embodiments the rufinamide does not cause at least one of drowsiness, lassitude, or alteration of mental and physical capabilities.

In certain embodiments the rufinamide is administered to the patient before or immediately after a traumatic event.

In certain embodiments the at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI). In certain embodiments the rufinamide significantly changes a score on at least one of CAPS, CAPS-2, CAPS-CA, IES, IES-R, CGI, CGI-S, CGI-I, DGRP, DGRP-I, HAM-A, SI-PTSD, PTSD-I, PSS-I, MADRS, BDI, HAM-D, RHRSD, MDI, GDS-30, and CDI. In certain embodiments the rufinamide significantly reduces an endpoint score compared to a baseline score on at least one of CAPS, CAPS-2, IES, IES-R, and HAMA. In certain embodiments the rufinamide significantly increases the proportion of responders on the CGI-I having CGI-I scores of at least one of 1 (very much improved) and 2 (much improved). In certain embodiments the rufinamide increases the proportion of responders on the DGRP-I having a DGRP-I scores of at least one of 1 (very much improved) and 2 (much improved). In certain embodiments an overall score of at least 65 on at least one of the CAPS and the CAP-2 is indicative of post-traumatic stress disorder. In certain embodiments an overall score of at least 18 on HAM-A is indicative of anxiety disorder. In certain embodiments a score of at least 3 on at least one of the CGI-I and the DGRP-I is indicative of post-traumatic stress disorder.

Also provided are methods of treating kindling in a patient. The methods include administering to the patient a therapeutically effective amount of rufinamide. In certain embodiments the rufinamide reduces the incidence of at least one of epilepsy and post-traumatic stress disorder in the patient.

In certain embodiments the methods further include coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the rufinamide reduces at least one of the frequency and intensity of at least one sign of the post-traumatic stress disorder in the patient. In certain embodiments the rufinamide reduces at least one of the frequency and intensity of at least one symptom of the post-traumatic stress disorder in the patient. In certain embodiments the rufinamide reduces at least one of the frequency and intensity of at least one symptom cluster of the post-traumatic stress disorder in the patient, wherein the symptom cluster is selected from re-experiencing/intrusion, avoidance/numbing, and hyperarousal. In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of treating post-traumatic stress disorder in a patient. The methods include diagnosing the patient with post-traumatic stress disorder; administering to the patient a therapeutically effective amount of rufinamide; assessing at least one of sign, symptom, and symptom cluster of post-traumatic stress disorder; and determining that the post-traumatic stress syndrome is improved if the rufinamide reduces at least one of sign, symptom, and symptom cluster of post-traumatic stress disorder.

In certain embodiments the methods further include coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the rufinamide reduces at least one of the frequency and intensity of at least one sign of the post-traumatic stress disorder in the patient. In certain embodiments the rufinamide reduces at least one of the frequency and intensity of at least one symptom of the post-traumatic stress disorder in the patient. In certain embodiments the rufinamide reduces at least one of the frequency and intensity of at least one symptom cluster of the post-traumatic stress disorder in the patient, wherein the symptom cluster is selected from re-experiencing/intrusion, avoidance/numbing, and hyperarousal. In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of improving resilience in a patient. The methods include administering a therapeutically effective amount of rufinamide. In certain embodiments the methods further include coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the rufinamide reduces at least one of the frequency and intensity of at least one sign of the post-traumatic stress disorder in the patient.

In certain embodiments the rufinamide reduces at least one of the frequency and intensity of at least one symptom of the post-traumatic stress disorder in the patient.

In certain embodiments the rufinamide reduces at least one of the frequency and intensity of at least one symptom cluster of the post-traumatic stress disorder in the patient, wherein the symptom cluster is selected from re-experiencing/intrusion, avoidance/numbing, and hyperarousal.

In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of diagnosing post-traumatic stress disorder in a patient. The methods include administering to the patient a therapeutically effective amount of rufinamide and assessing at least one of sign, symptom, or symptom cluster of post-traumatic stress disorder; and diagnosing post-traumatic stress disorder in the patient if the rufinamide reduces at least one of sign, symptom, and symptom cluster of post-traumatic stress disorder. In certain embodiments the patient is a child, adolescent, or adult.

Various scales can assess post-traumatic stress disorder (PTSD) and the effect of rufinamde and other therapies on the treatment and prevention of the disorder. These are, for example and without limitation, Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD

Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI). These measures generally are assessed by interviews or questionnaires. In certain embodiments, not all the parts of a scale are administered. In certain embodiments, the scales are used for diagnosing and assessing signs, symptoms, associated symptoms, or impact on daily life of PTSD. In certain embodiments, one or more scales are used to diagnose, assess, or confirm post-traumatic stress disorder in a patient. In certain embodiments, scales will measure signs, symptoms, symptom clusters by scoring at least one of the frequency and intensity of the signs, symptoms, or symptom clusters.

Examples of scales for post-traumatic stress disorder assessment are versions of CAPS, including CAPS, CAPS-1, and CAPS-2, which score 17 core PTSD symptoms with these items:

1. Recurrent and intrusive trauma recollections

2. Distress when exposed to trauma reminders

3. Acting or feeling as if event were recurring

4. Recurrent and distressing dreams of event

5. Efforts to avoid thoughts or feelings

6. Efforts to avoid activities or situations

7. Inability to recall trauma or trauma aspects

8. Markedly diminished interest in significant activities

9. Feelings of detachment or estrangement from others

10. Restricted range of affect

11. Sense of a foreshortened future

12. Difficulty falling or staying asleep

13. Irritability or outbursts of anger

14. Difficulty concentrating

15. Hypervigilance

16. Exaggerated startle response

17. Physiologic reactivity

Questions also target the impact of symptoms on social and occupational functioning or daily life, improvement in symptoms since a previous CAPS administration, overall response validity, overall PTSD severity, and frequency and intensity of associated symptoms. These items are:

18. Impact on Social Functioning

19. Impact on Occupational Functioning

20. Global Improvement (since earlier measurement occasion)

21. Rating Validity

22. Global Improvement

23. Guilt over acts committed or omitted

24. Survivor Guilt

25. Homicidality

26. Disillusionment with authority

27. Feelings of hopelessness

28. Memory Impairment

29. Sadness and depression

30. Feelings of being overwhelmed

To assess the frequency of symptoms, interviewers follow standard questions, clarifying or rephrasing as needed. Standard questions, by way of example and without limitation, are: Have you ever had unwanted memories of the traumatic event? What were they like? What did you remember? If the question requires rephrasing, the interviewer can ask a question such as: Did they ever occur while you were awake or only in dreams? or How often have you had these memories in the past month (week)? A score of 0 indicates a frequency of never, 1 indicates once or twice, 2 indicates once or twice a week, 3 indicates several times a week, and 4 indicates daily of almost every day.

To assess the intensity of symptoms, an interviewer may ask standard questions such as by way of example and without limitation: How much distress or discomfort did these memories cause you? Were you able to put them out of your mind and think about something else? How hard did you have to try? How much did they interfere with your life? A score of 0 indicates none, 1 indicates mild, minimal distress or disruption of activities, 2 indicates moderate, distress clearly present but still manageable, some disruption of activities, 3 indicates severe, considerable distress, difficulty dismissing memories, marked disruption of activities, and 4 indicates extreme, incapacitating distress, cannot dismiss memories, unable to continue activities.

In certain embodiments the scoring rule used counts a symptom as present if it has a frequency of at least 1 and an intensity of at least 2. In other embodiments severity scores are calculated by summing the frequency and intensity ratings for each symptom.

In certain embodiments, a total or overall score of all items on a version of CAPS is calculated. In certain embodiments, a total score for each symptom cluster is calculated. In certain embodiments, a total score for core symptoms of PTSD is calculated. In certain embodiments, an endpoint score is compared to a baseline score to determine the change in severity of post-traumatic stress disorder. In certain embodiments, a significant reduction of an endpoint score compared to a baseline score is considered improvement of PTSD. In certain embodiments, an overall score on CAPS, CAPS-1, CAPS-2, or CAPS-CA greater than 65 is indicative of PTSD.

Another example is the IES which assesses 15 items: 7 items measure intrusive symptoms and 8 items measure avoidance symptoms. The self assessed items ask how frequently each of the following comments are true: I thought about it when I didn't mean to, I avoided letting myself get upset when I thought about it or was reminded of it, I tried to remove it from memory, I had trouble falling asleep or staying asleep because of pictures or thoughts about it that came into my mind, I had waves of strong feelings about it, I had dreams about it, I stayed away from reminders of it, It felt as it hadn't happened or wasn't real, I tried not to talk about it, Pictures about it popped into my mind, Others things kept making me think about it, I was aware that I still had a lot of feelings about it, but I didn't deal with them, I tried not to think about it, Any reminder brought back feelings about it, and My feelings were kind of numb. The items are generally rated on a four point scale: 0 (not at all), 1 (rarely), 3 (sometimes), and 5 (often). The total of the scores provide an overall assessment of the severity of the symptoms or overall subjective stress. It has been suggested that a score from 0 to 8 is in the subclinical range, 9-25 is in the mild range, 26-43 is in the moderate range, and greater than 44 is in the severe range of stress.

In certain embodiments, a total or overall score of all items on IES is calculated. In certain embodiments, a total score for each symptom cluster is calculated. In certain embodiments, an endpoint score is compared to a baseline score to determine the change in severity of PTSD. In certain embodiments, a reduction of an endpoint score by 30% compared to a baseline score is considered improvement of PTSD.

The IES-R, a revision of the IES, changed the IES by splitting the original IES item, I had trouble falling asleep or staying asleep into two items: I had trouble falling asleep and I had trouble staying asleep and by adding six items to the IES items. These additional items are: I felt irritable and angry, I was jumpy and easily startled, I found myself acting or feeling as though I was back at that time, I had trouble concentrating, Reminders of it caused me to have physical reactions, such as sweating, trouble breathing, nausea, or a pounding heart, and I felt watchful or on guard. The scoring system also changed to 0 (not at all), 1 (a little bit), 2 (moderately), 3 (quite a bit), and 4 (extremely).

In certain embodiments, a total or overall score of all items on IES-R is calculated. In certain embodiments, a total score for each symptom cluster is calculated. In certain embodiments, an endpoint score is compared to a baseline score to determine the change in severity of post-traumatic stress disorder. In certain embodiments, a significant reduction of an endpoint score compared to a baseline score on the IES-R is considered improvement of post-traumatic stress disorder.

In the DGRP-I scale, the effectiveness of rufinamide in treating post-traumatic stress disorder can be assessed by measuring the increase in the proportion of responders on the DGRP-I having a DGRP-I of 1 (very much improved) or 2 (much improved). In certain embodiments, a score of at least 3 on the DGRP-I is indicative of post-traumatic stress

In the CGI, the effectiveness of rufinamide to treat post-traumatic stress disorder can be assessed by the CGI-S, CGI-I, and efficacy index. For example, in certain embodiments, an increase in the proportion of responders on the CGI-I having a CGI-I of 1 (very much improved) or 2 (much improved) after treatment indicates that the treatment is effective. In certain embodiments, a score of at least 3 on the CGI-I is indicative of post-traumatic stress disorder. In certain embodiments, the efficacy index on the CGI can measure the efficacy of rufinamide for treatment of post-traumatic stress disorder.

In HAMA-A, to assess anxiety or post-traumatic stress disorder, generally a total or overall score of all items on HAM-A is calculated. In certain embodiments, an endpoint score is compared to a baseline score on HAM-A to determine the change in severity of anxiety and post-traumatic stress disorder. In certain embodiments, a significant reduction of an endpoint score compared to a baseline score on HAM-A is considered improvement of anxiety and post-traumatic stress disorder. In certain embodiments, an overall score on HAM-A of at least 18 is indicative of anxiety and post-traumatic stress disorder.

Kindling is a mechanism that can contribute to neurological diseases including but not limited to post-traumatic stress disorder and epilepsy. Kindling refers to low level stimulation that sensitizes animals and triggers a disease state. In certain embodiments, low level stimulation is electrical or chemical stimulation. In a preclinical setting, kindling can be produced by repeated stimulation of various brain regions. In some settings, even after stimulation has ceased, cells continue to discharge. In some cases, seizures and convulsions can result. By comparing the effects of rufinamide on kindled animals with control animals that have not been stimulated, the therapeutic use of rufinamide on disease caused by kindling can be evaluated.

In general, rufinamide or a pharmaceutically acceptable derivative will be administered in therapeutically effective amounts, either singly or in combination with another therapeutic agent. The pharmaceutical compositions will be useful, for example, for the treatment of post-traumatic stress disorder.

Pharmaceutically acceptable derivatives include acids, bases, enol ethers, and esters, esters, hydrates, solvates, and prodrug forms. The derivative is selected such that its pharmokinetic properties are superior with respect to at least one characteristic to the corresponding neutral agent. The rufinamide may be derivatized prior to formulation.

A therapeutically effective amount of rufinamide or a pharmaceutically acceptable derivative may vary widely depending on the severity of the post-traumatic stress disorder, the age and relative health of the subject, the potency of the compound used and other factors. In certain embodiments a therapeutically effective amount is from about 0.1 milligram per kg (mg/kg) body weight per day to about 50 mg/kg body weight per day. In other embodiments the amount is about 1.0 to about 10 mg/kg/day. Therefore, in certain embodiments a therapeutically effective amount for a 70 kg human is from about 7.0 to about 3500 mg/day, while in other embodiments it is about 70 to about 700 mg/day.

One of ordinary skill in the art of treating such diseases will be able to ascertain a therapeutically effective amount of rufinamide for post-traumatic stress disorder without undue experimentation and in reliance upon personal knowledge and the disclosure of this application. In general, by way of example and without limitation, rufinamide will be administered as pharmaceutical compositions by one of the following routes: oral, systemic (e.g., transdermal, intranasal or by suppository) or parenteral (e.g., intramuscular, intravenous or subcutaneous). Compositions can, by way of example and without limitation, take the form of tablets, pills, capsules, semisolids, powders, sustained release formulations, solutions, suspensions, elixirs, aerosols, or any other appropriate composition and are comprised of, in general, rufinamide in combination with at least one pharmaceutically acceptable excipient. Acceptable excipients are, by way of example and without limitation, non-toxic, aid administration, and do not adversely affect the therapeutic benefit of the compound. Such excipient may be, for example, any solid, liquid, semisolid or, in the case of an aerosol composition, gaseous excipient that is generally available to one of skill in the art.

Solid pharmaceutical excipients include by way of example and without limitation starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from for example and without limitation water, ethanol, glycerol, propylene glycol and various oils, including those of petroleum, animal, vegetable or synthetic origin (e.g., peanut oil, soybean oil, mineral oil, sesame oil, etc.). Preferred liquid carriers, particularly for injectable solutions, include by way of example and without limitation water, saline, aqueous dextrose and glycols. Compressed gases may be used to disperse the compound in aerosol form. Inert gases suitable for this purpose are by way of example and without limitation nitrogen, carbon dioxide, nitrous oxide, etc.

The pharmaceutical preparations can by way of example and without limitation, moreover, contain preservatives, solubilizers, stabilizers, wetting agents, emulsifiers, sweeteners, colorants, flavorants, salts for varying the osmotic pressure, buffers, masking agents or antioxidants. In certain embodiments, they can contain still other therapeutically valuable substances. Other suitable pharmaceutical carriers and their formulations are described in A. R. Alfonso Remington's Pharmaceutical Sciences 1985, 17th ed. Easton, Pa.: Mack Publishing Company.

The amount of rufinamide in the composition may vary widely depending for example, upon the type of formulation, size of a unit dosage, kind of excipients and other factors known to those of skill in the art of pharmaceutical sciences. In general, the final composition will comprise from 10% w to 90% w of the compound, preferably 25% w to 75% w, with the remainder being the excipient or excipients. Preferably the pharmaceutical composition is administered in a single unit dosage form for continuous treatment or in a single unit dosage form ad libitum when relief of symptoms is specifically required.

The rufinamide or a pharmaceutically acceptable derivative thereof is administered simultaneously with, prior to, or after administration of one or more of the above agents.

The invention is further illustrated by the following non-limiting examples.

### EXAMPLES

### Example 1

A clinical study is performed to demonstrate the efficacy and tolerability of rufinamide in the treatment of post-traumatic stress disorder (PTSD).

The research design includes an 8-week randomized, double-blind, placebo-controlled treatment trial of rufinamide for the treatment of PTSD.

After signing an informed consent and meeting inclusion/exclusion criteria, the patient is randomized to receive either rufinamide or placebo for the 8-week duration. Patients' symptoms, side effects and compliance is assessed bi-weekly.

Based on symptomotology and occurrence of side effects, the investigator may increase the medication in 200-400 mg increments, as tolerated, until a maximum therapeutic benefit is achieved, not to exceed 45 mg/kg/day. The dosing is once per day unless twice per day is better tolerated. Compliance is assessed by pill count at week 4 and week 8.

Efficacy is measured by the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Åsberg Depression Rating Scale (MADRS)
Treatment Outcome PTSD rating scale (TOP-8)

The subject inclusion criteria are:
Diagnosis of PTSD that is confirmed by Mini International Neuropsychiatric Interview (MINI) and CAPS
Age 13 or older
No substance abuse or dependence for the previous 4 weeks (except for nicotine and caffeine)
Free of psychotropic medication for 2 weeks (except 4 weeks for fluoxetine)
Clinically normal physical and laboratory examination (Liver function tests (LFTs) up to 2.5 times the normal limit is allowed.)
Women of childbearing potential must be using medically approved methods of birth control such as a condom, birth control pill, Depo-Provera, or diaphragm with spermicides
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to rufinamide
Unstable general medical conditions
Score>6 on Question #10 of MADRS regarding suicidal ideation
Women who are pregnant, planning to become pregnant or breastfeed during the study

Fulfillment of only one exit criterion is needed to exit the study. Exit criteria are:
Completion of the study
Severe and intolerable side effects to rufinamide or placebo treatment
Acute development of suicidal ideation, homicidal ideation or psychotic symptoms
Worsening of symptoms as measured by a score of 7 (very much worse) on CGI-I
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 2

A clinical study is performed to demonstrate the efficacy and tolerability of rufinamide in the prevention of PTSD.

The research design includes an open-ended randomized, double-blind, placebo-controlled treatment trial of rufinamide for the prevention of PTSD. After signing an informed consent and meeting inclusion/exclusion criteria, patients are randomized to receive either rufinamide versus placebo for the 8-week duration. Patients' symptoms, side effects and compliance are assessed bi-weekly.

Based on symptomatology and occurrence of side effects, the investigator can increase the medication in 200-400 mg increments, as tolerated, until a maximal tolerated dose is achieved, not to exceed 45 mg/kg/day The dosing is once per day unless twice per day is better tolerated. Compliance is assessed by pill count at week 4 and week 8.

Efficacy is measured by the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Åsberg Depression Rating Scale (MADRS)
Treatment Outcome PTSD rating scale (TOP-8)
Diagnostic and Statistical Manual IV (DSM-IV)

The subject inclusion criteria are:
Absence of PTSD, confirmed by MINI and CAPS
Age 13 or older
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The exclusion criteria are:
History of PTSD
Lifetime history of bipolar I, psychotic, or cognitive disorders
Actively suicidal, homicidal, or psychotic
History of sensitivity to rufinamide
Unstable general medical conditions
Score>6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to rufinamide or placebo treatment
Acute development of suicidal ideation, homicidal ideation or psychotic symptoms
Appearance of signs or symptoms compatible with a diagnosis of PTSD.
Participant's explicit request to exit the study
The need for additional psychotropic drugs, other than the study drug or adjunctive medication as specified in the protocol, for the control of the subjects psychiatric symptoms.
The subject becomes pregnant during the course of the study.
Investigator's judgment that it is no longer in the best interest of the patient to continue in the study.

### Example 3

A clinical study is conducted to demonstrate the efficacy and tolerability of rufinamide combination therapy in the treatment of PTSD.

The research design includes an 8-week randomized, double-blind, placebo-controlled treatment trial of rufinamide for the treatment of PTSD. After signing an informed consent and meeting inclusion/exclusion criteria, the patient is randomized to receive either rufinamide or placebo for 8-week duration. Patients can also receive therapeutically effective doses of prazosin, valproate, carbamazepine, or topiramate in combination with rufinamide or placebo.

During the study a pharmacist maintains the randomization log and verifies the order for the placebo or rufinamide in look-a-like tablets. Patients' symptoms, side effects and compliance is assessed bi-weekly. Based on symptomatology and occurrence of side effects, the investigator increases the medication in 200-400 mg increments, as tolerated, until a maximum therapeutic benefit is achieved, not to exceed 45 mg/kg/day. The dosing is once per day unless twice per day is better tolerated. Compliance is assessed by pill count at week 4 and week 8.

Efficacy is measured by the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Åsberg Depression Rating Scale (MADRS)
Treatment Outcome PTSD rating scale (TOP-8)

The subject inclusion criteria are:
Diagnosis of PTSD, confirmed by MINI and CAPS
Age 13 or older
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to rufinamide
Unstable general medical conditions
Score>6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to rufinamide or placebo treatment
Acute development of suicidal ideation, homicidal ideation or psychotic symptoms
Symptoms worsen as measured by a Score of 7 (very much worse) on CGI-I
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 4

A clinical study is performed to demonstrate the efficacy and tolerability of rufinamide in the treatment of PTSD in children.

The research design includes an 8-week randomized, double-blind, placebo-controlled treatment trial of rufinamide for the treatment of PTSD.

After signing an informed consent and meeting inclusion/exclusion criteria, the patient is randomized to receive either rufinamide or placebo for an 8-week duration. During the study a pharmacist maintains the randomization log and verify the order for the placebo or rufinamide in look-a-like tablets. Patients' symptoms, side effects and compliance are assessed bi-weekly.

Based on symptomatology and occurrence of side effects, the investigator can increase the medication in 200-400 mg increments, as tolerated, until a maximum therapeutic benefit is achieved, not to exceed 45 mg/kg/day. The dosing is once per day unless twice per day is better tolerated. Compliance is assessed by pill count at week 4 and week 8.

Efficacy is measured by the following assessment scales:
Global Assessment of Functioning (GAF)
Clinician Administered PTSD Scale (CAPS)
Clinician Administered PTSD Scale (CAPS-CA)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Åsberg Depression Rating Scale (MADRS)
Treatment Outcome PTSD rating scale (TOP-8)

The subject inclusion criteria are:
Diagnosis of PTSD, confirmed by MINI and CAPS
Age 12 or younger
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to rufinamide
Unstable general medical conditions
Score>6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to rufinamide or placebo treatment
Acute development of suicidal ideation, homicidal ideation or psychotic symptoms
Symptoms worsen as measured by a Score of 7 (very much worse) on CGI-I.
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Examples 5

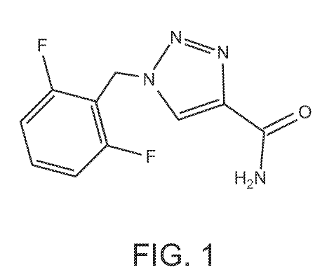

There remains a paucity of data investigating the anxiolytic potential of this rufinamide. Therefore the aim of the present study was to test the effect of rufinamide (FIG. 1, chemical structure of rufinamide) with regard to a possible facilitation of social behavior (anxiolytic-like effect) using the social exploration test in rats as a paradigm. The basis of this test is that when an adult “resident” rat is kept in social isolation for two weeks, its natural tendency to establish territorial defensive reactions is increased. A young “intruder” rat, placed into the home cage of an adult “resident” rat, shows fear and its level of social exploration is minimal. Anxiolytic compounds such as chlordiazepoxide or diazepam have been found to reduce the intruder's fear and to increase its tendency to scrutinize the resident partner. Male adult Sprague Dawley rats (Ra238, OFA/IC strain, Iffa Credo, France) (“residents”) weighing 350-400 g and young Lister Hooded rats (li/ho, Harlan, The Netherlands) (“intruders”) weighing 100-120 g were used. “Intruders” were housed in pairs and the “residents” were individually housed for two weeks before the test in plastic cages (Macrolon, 42×26×15 cm). All animals lived in a room lit from 6 a.m. until 6 p.m. and had always free access to standard food and water. All observations were made during the light phase (8 a.m. to 11a.m.) in the home cage of the “resident” (see above), the floor of which was covered with sawdust.

Test-compounds were suspended in 0.5% methyl cellulose (Methocel) and administered orally (5 ml/kg) to the “intruder” rat. Rufinamide (rufinamide) was administered at doses of 0.3, 1, 3, 10, 30 or 100 mg/kg p.o. Control rats received 0.5% methyl cellulose (Methocel) and an additional group (positive standard) was treated with 5 mg/kg p.o. chlordiazepoxide-HCl (CDZ, RBI, Massachusetts, USA), diluted in the same vehicle. All rats were tested 1 hour after oral application.

Pairs consisting of one “intruder” rat and one “resident” rat were assigned at random to one of the experimental or the control groups. In each pair only the “intruder” was orally treated before being placed into the home cage of a “resident” animal. The duration of active approach behaviors of the “intruder” rat (sniffing, anogenital exploration, nosing, grooming, licking, playing) towards the “resident” was manually registered and cumulatively recorded over a period of 5 minutes.

Comparisons between groups were performed using a one way analysis of variance (ANOVA), followed when appropriate by the posthoc Dunnett's test (multiple comparison of different doses versus the vehicle-treated rats). Values of p<0.05 were considered as statistically significant.

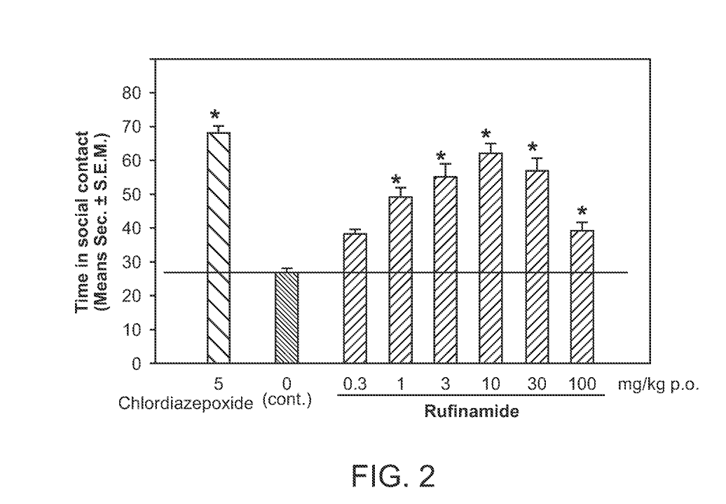

The analysis of variance indicated a significant intergroup difference [F(7,88)=21.759; p<0.001] and the posthoc comparisons revealed that the 1, 3, 10, 30 and 100 mg/kg p.o. doses of rufinamide, as well as the positive standard chlordiazepoxide (5 mg/kg p.o.) significantly increased the duration of social contacts of an “intruder” toward a “resident” rat (see FIG. 2). The dose of 0.3 mg/kg p.o. rufinamide did not induce any significant change in the time spent in social contact.

### Example 6

Rufinamide, a Na-channel blocker and anti-epileptic agent, and carbamazepine, an anticonvulsant employed to treat bipolar disorder, were given to male OF-1 “intruder” in oral-doses of 1/3/10 mg/kg, 1 hr before the animals were confronted with an individually-housed, aggressive, male OF-1 resident mouse. Ethological analysis showed that rufinamide significantly increased the frequency of non-social activity i.e. exploration of the cage, in a dose-related manner, this being its main effect. Rufinamide additionally increased social investigation directed towards the residents, albeit, in a bell-shaped manner with only 3 mg/kg attaining statistical significance. Further analysis showed that rufinamide significantly and dose-relatedly shortened the duration of flight postures and reduced acts of escape. This profile indicates that in these mice rufinamide exerts a weak sociotropic (approach-promoting) action which is largely masked by stimulant component. The fact that the total number of elements measured during the 6 minute encounter was increased in a dose-related manner attests to the stimulant action of rufinamide. In contrast to rufinamide, carbamazepine exerted a clear, dose-related increase in both the frequency and time the intruders spent investigating their opponents. This increase in social investigation (sociotropic effect) was accompanied by a decrease in the frequency and duration of defensive ambivalence probably through competitive inhibition. No signs of motor stimulation were detected.

The results of these studies raise caution with respect to the intention of employing rufinamide for treating bipolar disorder since manic episodes would be aggravated by its stimulant component. However, rufinamide's mild sociotropic action combined with its stimulant component could indicate its use for the treatment of attention-deficit-hyperactivity (ADHD) disorder and perhaps other disorders of attention e.g. autism. Ongoing studies with methylphenidate in our laboratory are intended to throw more light on this proposal.

The Intruder test duplicates a territorial encounter between a territory holder (aggressive male) and a subordinate “intruder” mouse. Intruders exhibit high levels of defensive behaviour but low levels of aggression and approach-oriented behaviours and are a model of a flight-induced state of social withdrawal. We have shown that carbamazepine reverses social withdrawal in this test. In this test the intruders receive the treatment whereas the residents remain untreated. (Dixon, A. K., Huber, C., Lowe, D. J. Clin. Psychiatry. 55 (9) Suppl B, 4-7 (1994)). For purposes of this study, rufinamide and carbamazepine were given orally to intruder mice 1 hr before being introduced singly to aggressive residents using doses of ⅓ and 10 mg/kg.

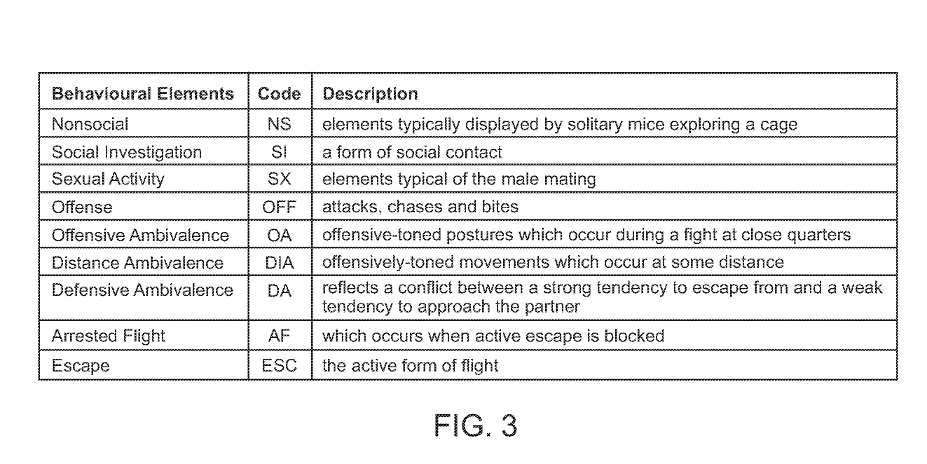

Over 60 ethological elements describing the social and non-social behaviour of mice based on the ethogram originally proposed by Grant and Mackintosh (Grant, E. C. Mackintosh, J. H. Behaviour 21: 246-259 (1963) ) and classified according to 9 behavioural categories (Dixon, A. K., Huber, C., Fisch, H. U. Adv. Study. Behaviour. 19: 171-204 (1990)) form the rodent ethogram as used in this test (FIG. 3).

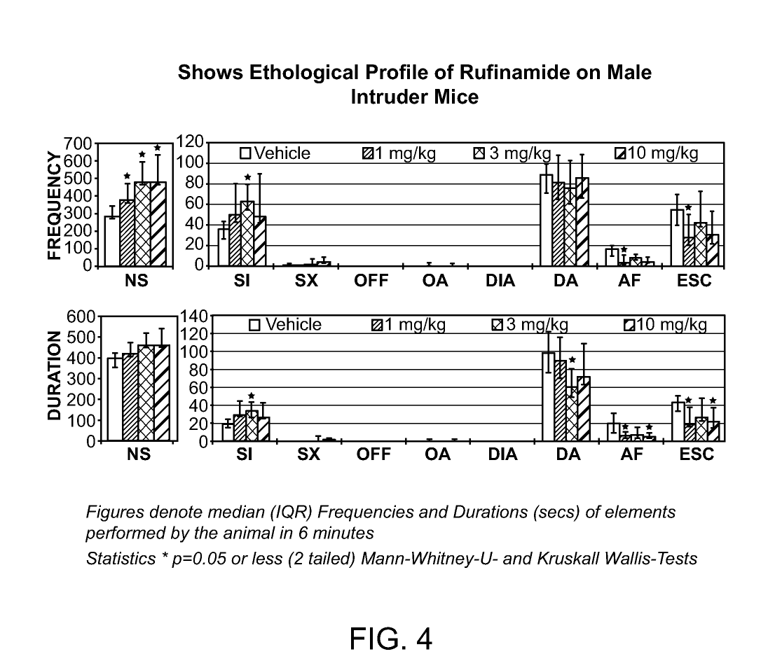

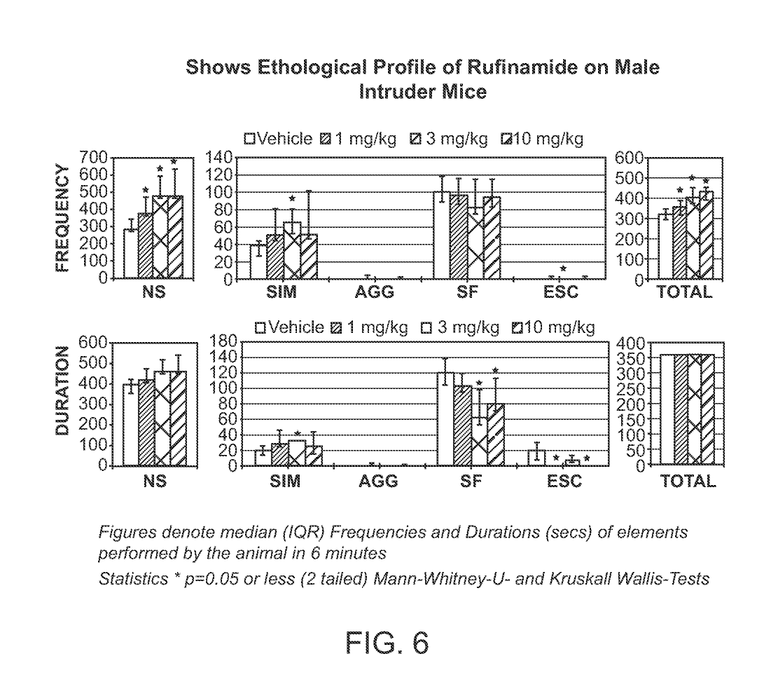

FIG. 4 shows the overall ethological profile of rufinamide on the intruder mice. Inspection of frequency changes indicate that rufinamide induced a significant, dose-related increase in non-social (NS) behaviour with the time spent in non-social activities only weakly following suit. Social investigation (SI) also increased, albeit in a bell-shaped manner with only changes at 3 mg/kg attaining significance. The duration of defensive ambivalence (DA), arrested flight (AF) and escape were significantly reduced but not uniformly. The frequencies of arrested flight and escape were only reduced significantly at 1 mg/kg. However, when defensive ambivalence and arrested flight were combined to give a measure of static flight (SF) elements, a significant shortening of the time spent in static flight postures was evident (FIG. 6) at 3 and 10 mg/kg. Overall, the total number of ethological elements, irrespective of their function, performed by the mice in the 6 minute encounter was increased in a dose-related manner.

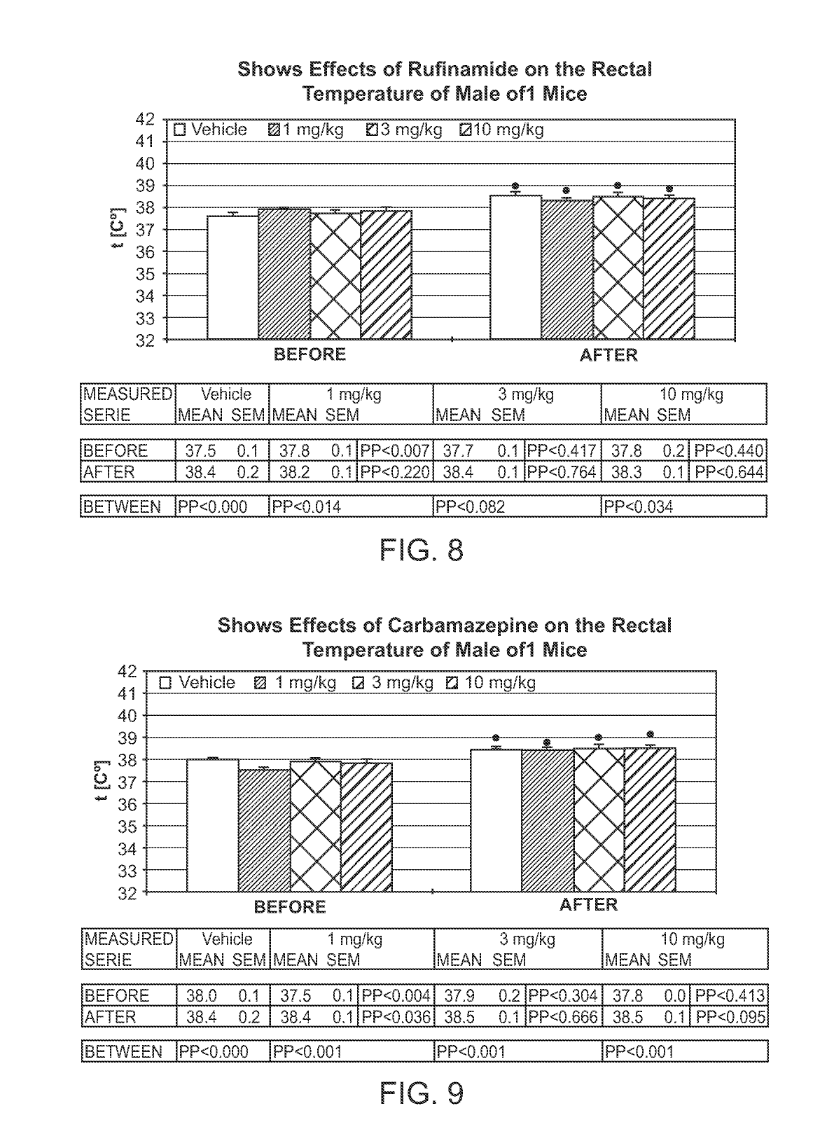

FIG. 8 shows that rufinamide did not alter basal body temperature nor alter the post-encounter hyperthermia of intruder mice.

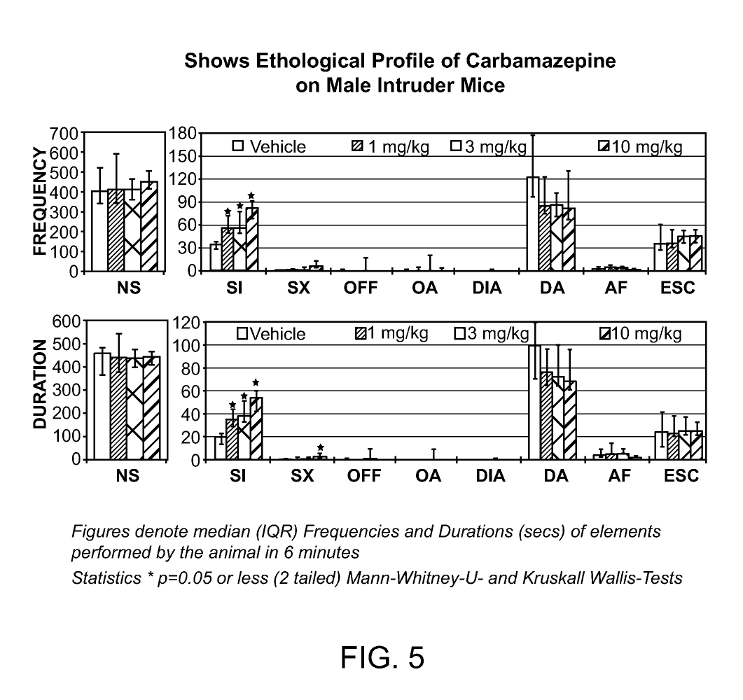

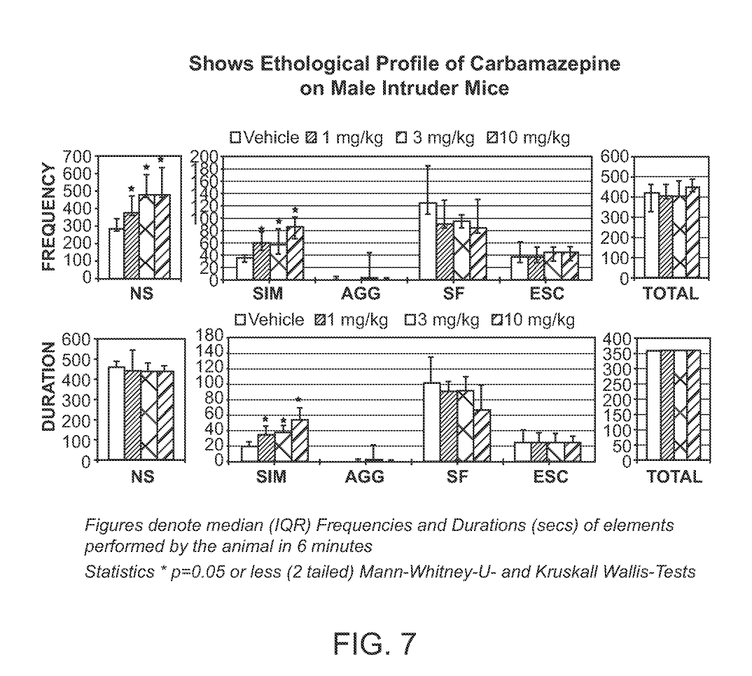

FIG. 5 shows the overall ethological profile of carbamazepine on the intruder mice. Inspection of the behavioural changes of the intruders indicates that carbamazepine induced a dose-related increase in social investigation with both changes in frequencies and durations attaining significance. Defensive ambivalence, the main defensive response of intruders from attacks, was reduced but, because of large variation did not attain significance. FIG. 7 shows that when expressed as static-flight elements, the dose-related reduction persisted but also did not attain significance. FIG. 7 also shows that combining elements of social investigation with those of mating behaviour to give an expression of overall social contact, (SIM), preserved the clear sociotropic action of this drug. In addition it is evident that non-social elements and total numbers of elements did not change after carbamazepine treatment.

FIG. 9 shows that carbamazepine did not alter basal body temperature nor alter the post-encounter hyperthermia of intruder mice.

Rufinamide clearly acted as a stimulant in this test. It elevated the total number of ethological elements performed during the encounter, the effects on non-social behaviour (exploration) being the main contributing factors subserving this effect. In addition it increased social investigation, albeit, in a bell-shaped manner. Flight behaviour, particularly flight-postures were decreased. Thus rufinamide appears to have a catadrasic (flight-attenuating) effects as well as a sociotropic (approach-promoting) action. The latter however, appears to be held in check by the stimulant action of rufinamide which is clearly the overiding behaviour property found in this study. The stimulant properties detected here are further confirmed by the findings in the half-enclosed platform test in which rufinamide (0.32-10 mg/kg p.o.) exerted a dose-related increase in exploratory elements. Carbamazepine, in contrast to rufinamide caused no stimulation of behaviour. Its main effect was a clear, dose-related increase in social investigation (sociotropic action) which intruders directed towards their aggressive opponents. The fact that a dose-related but variable reduction in defensive ambivalence was found in the absence of increases in non-social behaviour strongly suggests that the reduction in this form of flight behaviour was competitively induced. Of interest also, is that neither compound altered the basal and post-encounter body temperature in any way.

In terms of clues for their clinical utility, carbamazepine's proven capacity to reverse this form of social withdrawal fits well with its use in bipolar disorder. There are grounds for suspecting that a compound having a measure of sociotropic activity combined with a clear stimulant component could find use as a treatment for attention-deficit-hyperactivity disorder (ADHD) and perhaps other attentional disorders associated with social impairment e.g. autism.

### Example 7

Rufinamide and carbamazepine were compared in the half-enclosed platform (HEP) test. The test determines how drugs affect the exploratory behaviour of a mouse placed upon a novel, half-enclosed platform and gives information as to whether a drug is a stimulant, depressant or disinhibitory as with, for example the benzodiazepines which increase exploration of the platform. It is also useful for determining duration of action and dose-findings studies. The HEP is a further development of the simple elevated platform used by Kaesermann to assess anxiolytic properties of drugs (Ref. Käsermann H. P. (1986) Psychopharmacol. 89:31-37). In this study carbamazepine and rufinamide were tested in the same dose ranges i.e. 0.32/1/3.2 and 10 mg/kg p.o. of each were given to male OF-1 mice 1 hr before testing.

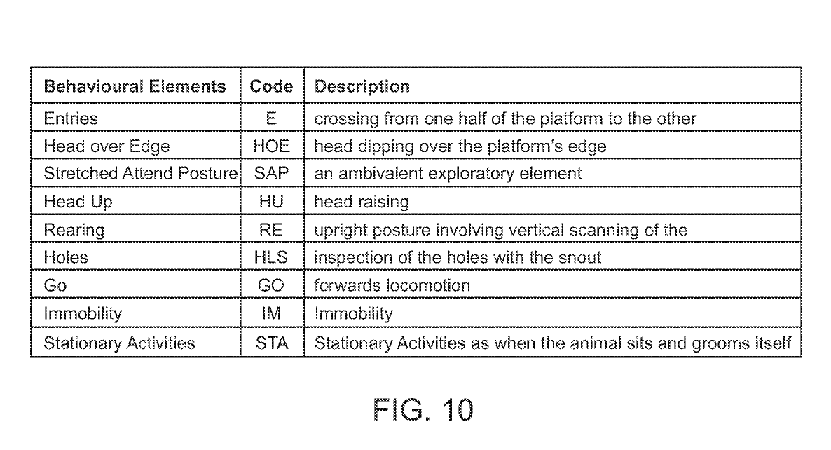

The ethogram used consists of 9 elements of exploratory behaviour two of which involve static postures i.e. immobility and stretched attend posture, the latter being an ambivalent element indicative of approach-avoidance conflict. These elements are immediately spontaneous and are not induced by either training or punishment procedures. Upright postures which are posturally demanding are useful to detect muscle relaxation e.g. rearing (FIG. 10).

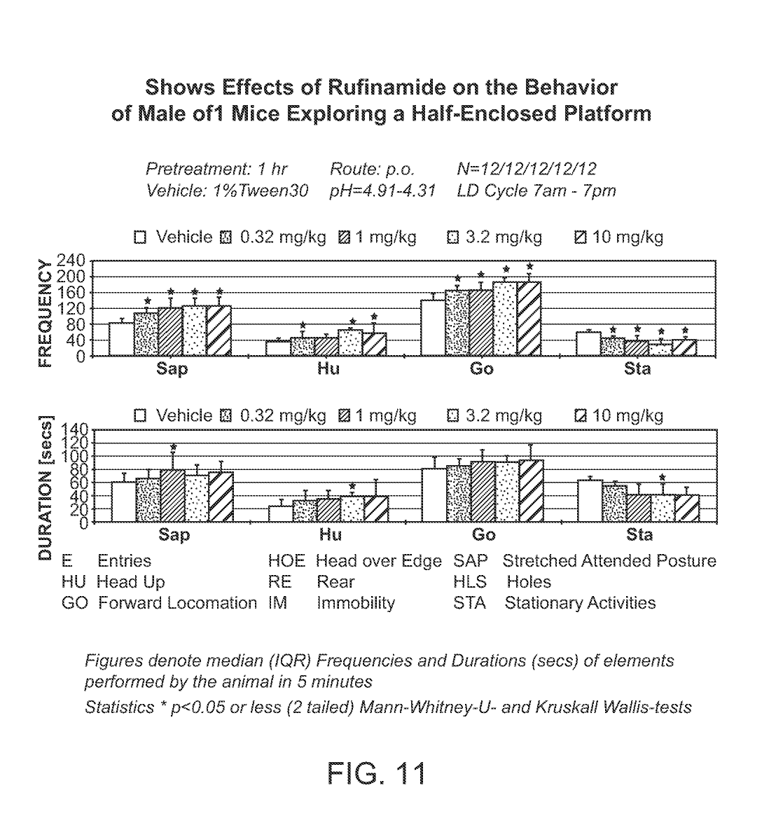

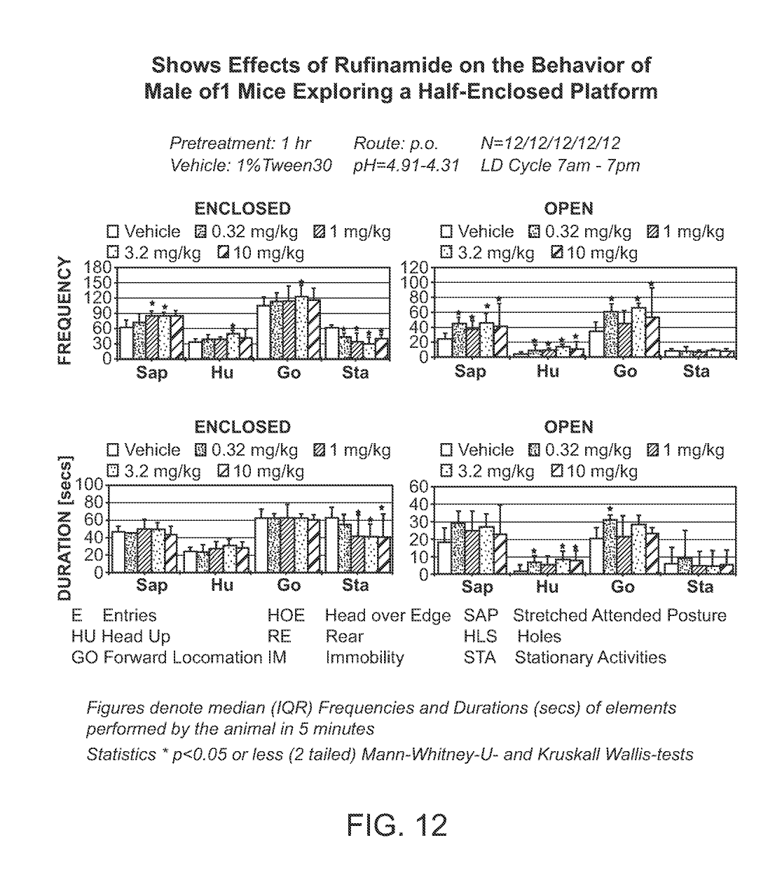

Reference to FIG. 11 shows that the exploratory elements stretched attend posture (sap), head raising (hu) and forward locomotion (go) were significantly increased by rufinamide in a dose-related manner. Frequencies of stationary elements (Sta: sitting still, inactivity) were decreased in a significant manner. Changes in durations followed suit but rarely attained statistical significance. FIG. 12 shows that when the effects of rufinamide were considered in terms of each half of the platform, most of the significant increases occurred in the open half of the platform whereas significant decreases in inactivity occurred in the enclosed half. Inactivity (sta) was shortened in the enclosed half whereas the duration of head raising increased in the open half.

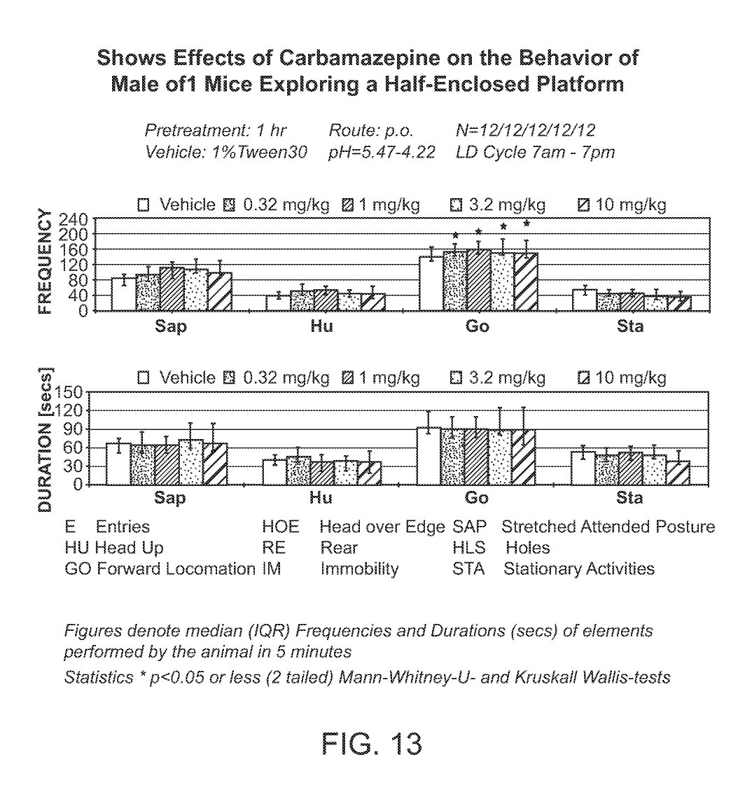

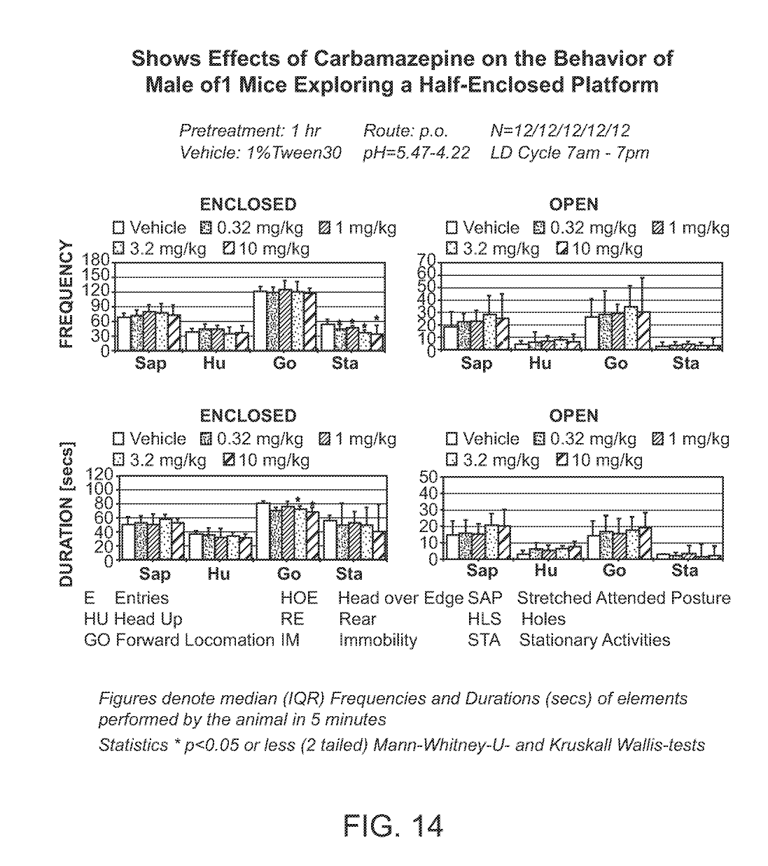

FIG. 13 shows the overall ethological profile after the mice received carbamazepine. No major changes in behaviour occurred. However, FIG. 14 shows that significant shortening of the time spent in forward locomotion (Go) in the enclosed half was evident at 3.2 and 10 mg/kg as well as a significant decrease in elements of stationary activity at 10 mg/kg. Although not attaining statistical significance, a general tendency for increased exploratory activity (Sap,Go) in the open half of the platform was discernible.

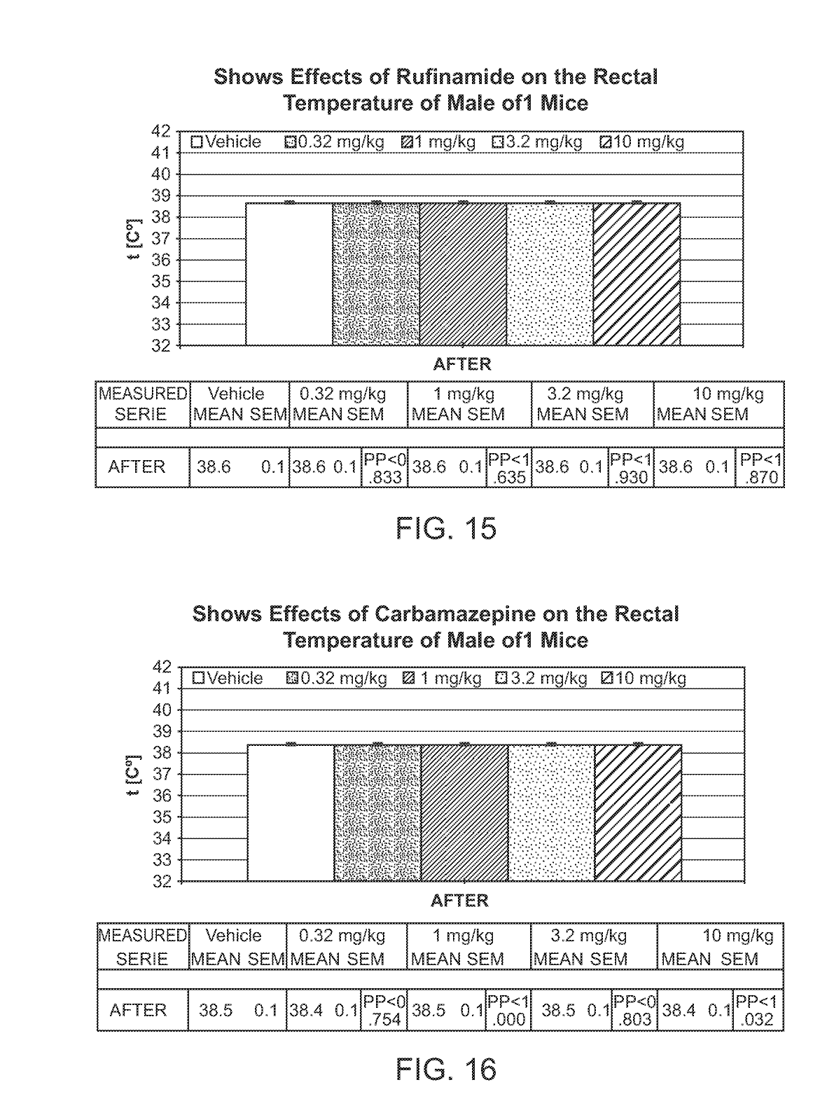

FIGS. 15 and 16 showed that neither rufinamide nor carbamazepine altered post trial rectal temperatures in the mice.

Rufinamide-treated mice performed significantly more exploratory elements in 5 minutes whereas the time spent in these activities was only marginally increased. This means that the animals were faster in executing switches in exploratory activities and shows them to be stimulated by rufinamide. However, since most of the activities occurred in the open half, the results indicate that rufinamide exerted a measure of behavioural disinhibition. In the HEP test, disinhibition is demonstrable with diazepam but not amphetamine.

The present results did not reveal any stimulant or disinhibiting effects on behaviour for equi-dosed carbamazepine although signs of a trend towards exploration of the open half of the platform were discernible. The results show that in the half-enclosed platform, rufinamide but not carbamazepine, exerts stimulant and possibly behavioural disinhibiting effects on exploratory activity in male mice.

### Example 8

Rufinamide was tested in the modified stress-induced model of anxiety. The stress-induced hyperthermia test in mice is a paradigm developed several years ago to model the expression of autonomic hyperactivity in anxiety (Lecci A, Borsini F, Volterra G, Meli A. (1990) Pharmacological validation of a novel model of anticipatory anxiety in mice. Psychopharmacology; 101:255-61.; Spooren W P, Schoeffter P, Gasparini F, Kuhn R, Gentsch C. (2002) Pharmacological and endocrinological characterisation of stress-induced hyperthermia in singly housed mice using classical and candidate anxiolytics (LY314582, MPEP and NKP608). Eur J Pharmacol; 435:161-70.;Zethof T J J, van der Heyden J A M, Tolboom J B M, Olivier B (1994) Stress-induced hyperthermia in mice: a methodological study. Phys. Behav. 55:109-115.). Mice who are mildly stressed (temperature taking is the stressor employed) exhibit an increase in body temperature which can be prevented by pretreatment with a variety of standard and putative anxiolytic agents such as benzodiazepines, HT1A receptor agonists, NK1 receptor antagonists and metabotropic glutamte5 receptor antagonists (Spooren W P, Schoeffter P, Gasparini F, Kuhn R, Gentsch C. (2002) Pharmacological and endocrinological characterisation of stress-induced hyperthermia in singly housed mice using classical and candidate anxiolytics (LY314582, MPEP and NKP608). Eur J Pharmacol; 435:161-70; Olivier B, Bouwknecht J A, Pattij T, Leahy C, van Oorschot R, Zethof T J (2002) GABA(A)-benzodiazepine receptor complex ligands and stress-induced hyperthermia in singly housed mice. Pharmacol Biochem Behav 72:179-188.

It offers many advantages over other anxiety tests in that it is fast, easy to carry out and is not hindered by either potential sedative or stimulant effects of compounds. This latter point is particularly relevant, as rufinamide has been reported to have stimulant-like properties in the half-enclosed platform in mice.

Male OF1/IC mice (MA205 Tiger code) were obtained several days prior to behavioural testing from Iffa Credo (L'Arbresle, France; 18-20 g) and housed in groups of 15 animals per cage (macrolon cages 42H26H15 cm, Type 4). The animals were individually housed (macrolon cage: 26H21H14 cm, Type 2) 24 h before testing. The day preceding the experiment, all cages were transferred to the testing room—and background noise (radio) was used during the light cycle. Both the animal room and the laboratory were temperature controlled and equipped with artificial illumination (L12:D12; lights on 06:00). Mice always had ad libitum access to water and food (Ecosan, Eberle Nafag A G, Gossau, Schweiz).

The test procedure for the modified stress-induced hyperthermia was adapted from van der Heyden et al. (1997). Rectal temperature was measured in each mouse twice, i.e. at t=0 min (T1) and t=+15 min (T2). The difference in temperature (T2-T1) was considered to reflect the stress-induced hyperthermia (SIH). Timepoints were based on previous experiments which showed that a T2-T1 interval of 15 min was optimal in terms of stress-induced hyperthermia. In addition, a comparison between T1 in vehicle-treated mice and in animals treated with a given dose of a test-compound was used, to measure whether a test-compound affected body temperature alone.

Rectal temperature was measured to the nearest 0.1° C. by an ELLAB instruments (Copenhagen, Denmark) thermometer Model DM 852 by inserting a lubricated thermistor probe model PRA-22002-A (ELLAB, Copenhagen, Denmark) (2.2 mm diameter) 20 mm into the rectum; the mouse was hand held at the base of the tail during this determination and the thermistor probe was left in place for 15 s. Chlordiazepoxide (CDZ) (10 mg/kg, p.o.) was used as a positive control in all studies as this dose has been shown from previous studies to elicit a maximal reversal of SIH response (Spooren W P, Schoeffter P, Gasparini F, Kuhn R, Gentsch C. (2002) Pharmacological and endocrinological characterisation of stress-induced hyperthermia in singly housed mice using classical and candidate anxiolytics (LY314582, MPEP and NKP608). Eur J Pharmacol; 435:161-70).

All drugs were made up fresh and dissolved in vehicle (0.5% methylcellulose) and administered per os (p.o.) in a volume of 10 ml/kg.

All data was analysed using a one-way analysis of variance (ANOVA) followed where appropriate by Dunnett test of multiple comparisons. The level of significance was set at p<0.05.

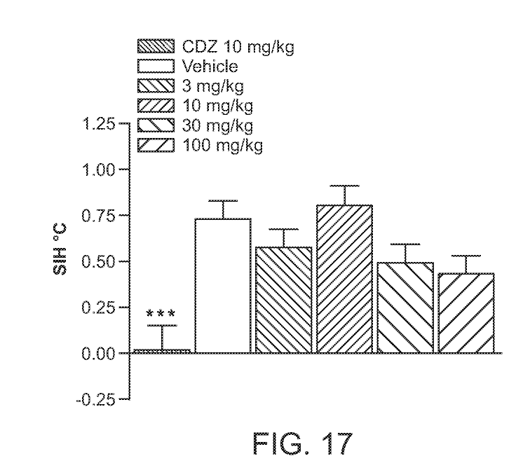

Rufinamide (3, 10, 30, 100 mg/kg, p.o.) and CDZ (10 mg/kg, p.o.) were tested in the SIH paradigm. SIH was assessed as the difference in body temperature 15 minutes after stress (the taking of rectal temperature is the stressor) compared to baseline temperature which was determined 60 minutes after subjects were treated p.o. with rufinamide, chlordiazepoxide (CDZ) or vehicle (methylcellulose). Bars represent mean and sem (n=12). *** p<0.001 vs. control. ANOVA revealed significant effect of treatment on the magnitude of the SIH response [F(5, 66)=6.39, p<0.001]. Post-hoc analysis revealed that only the positive control CDZ (10 mg/kg, p.o.) reversed significantly the SIH response (p<0.001) (FIG. 17). Although treatment with rufinamide (10-30-100 mg/kg) tended to decrease basal body temperature T1 [F(5, 66)=3.20, P=0.012], subsequent pair-wise comparisons revealed that neither rufinamide nor the positive control CDZ significantly altered baseline body temperature.

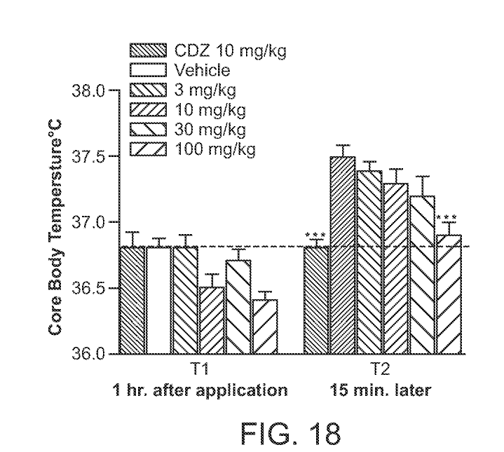

ANOVA indicated a significant effect of drug treatment on T2 [F(5, 66)=7.59, p<0.01]. Animals treated with both rufinamide at the highest dose (100 mg/kg, p.o.) or with CDZ (10 mg/kg, p.o.) had a significant lower body temperature than control animals (p<0.001) (FIG. 18). Body temperature was measured 60 minutes after treatment with rufinamide, CDZ, or vehicle (T1) and again 15 minutes later (T2). Bars represent mean and sem. *** denotes p<0.001 versus control.

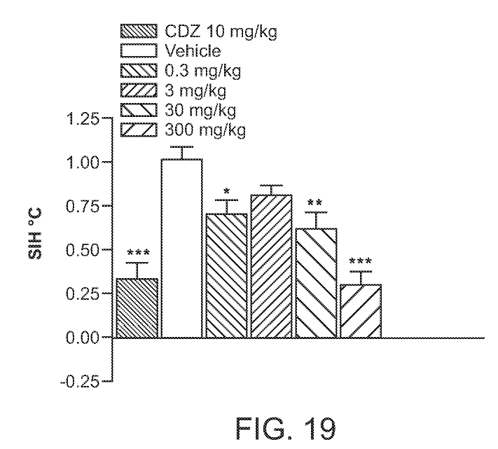

In order to expand the dose range tested rufinamide (0.3, 3, 30, 300 mg/kg, p.o.) and CDZ (10 mg/kg, p.o.) were tested in the SIH paradigm. ANOVA revealed significant effect of treatment on the magnitude of the SIH response [F(5, 66)=12.32, p<0.001]. Post-hoc analysis revealed that rufinamide at the doses of 0.3, 30 and 300 mg/kg, p.o. (p<0.05, p<0.01 and p<0.001, respectively) and CDZ at 10 mg/kg, p.o. (p<0.001) significantly attenuated the SIH response (FIG. 19). SIH was assessed as the difference in body temperature 15 minutes after stress (the taking of rectal temperature is the stressor) compared to baseline temperature which was determined 60 minutes after subjects were treated p.o. with rufinamide, chlordiazepoxide (CDZ) or vehicle (methylcellulose). Bars represent mean and sem (n=12). *** p<0.001 vs. control.

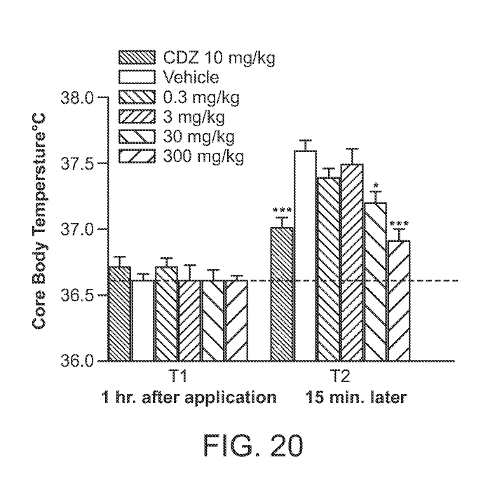

Neither rufinamide nor CDZ significantly modified the basal body temperature T1 [F(5, 66)=0.49, P=0.784]. However, there was a significant effect of drug treatment on T2 [F(5, 66)=7.4, P<0.01]. Post-hoc analysis revealed that animals treated with both rufinamide at the highest doses (30 and 100 mg/kg, p.o.) or with CDZ (10 mg/kg, p.o.) had a significant lower body temperature than control animals (FIG. 20). Body temperature was measured 60 minutes after treatment with rufinamide, CDZ, or vehicle (T1) and again 15 minutes later (T2). Bars represent mean and sem. *** denotes p<0.01 versus control.

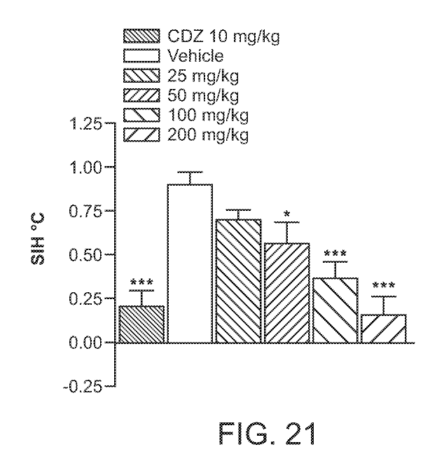

In order to confirm the anxiolytic-like effects of rufinamide observed in the second trial, rufinamide (25, 50, 100, 200 mg/kg, p.o.) and CDZ (10 mg/kg, p.o.) were tested in the SIH paradigm. ANOVA revealed an overall change in magnitude of the SIH response [F(5, 66)=9.84, p<0.001]. Rufinamide at the doses of 50 (p<0.05), 100 and 200 mg/kg, p.o. (p<0.001) and CDZ at 10 mg/kg, p.o. significantly reduced the SIH response (FIG. 21). SIH was assessed as the difference in body temperature 15 minutes after stress (the taking of rectal temperature is the stressor) compared to baseline temperature which was determined 60 minutes after subjects were treated p.o. with rufinamide, chlordiazepoxide (CDZ) or vehicle (methylcellulose). Bars represent mean and sem (n=12).

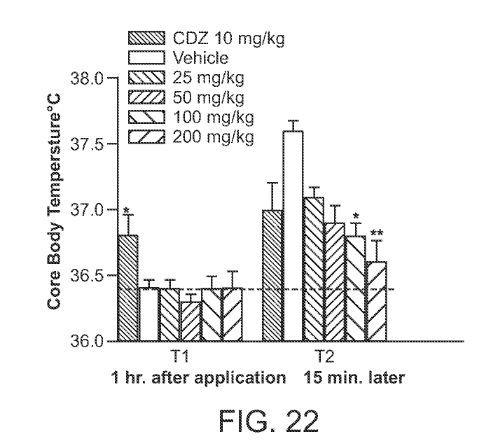

In this experiment, CDZ induced a slight but significant increase of basal body temperature T1 (p=0.033) [F(5, 66)=3.05, p=0.015]. In contrast, rufinamide (25-50-100-200 mg/kg, p.o.) did not modify this parameter. ANOVA indicated that here was a significant effect of drug treatment on T2 [F(5, 66)=3.40, p=0.009]. Post-hoc analysis revealed that animals treated with rufinamide at the highest doses (100 and 200 mg/kg, p.o.) had a significant lower body temperature than control animals. Due to the increase in T1 in animals treated with the positive control CDZ no stress-attenuating effect was seen when performing independent comparisons of T2 (FIG. 22). Body temperature was measured 60 minutes after treatment with rufinamide, CDZ, or vehicle (T1) and again 15 minutes later (T2). Bars represent mean and sem. ** denotes p<0.05 versus control.

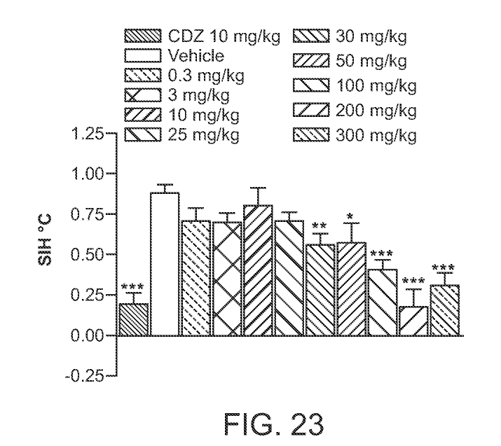

In order to get an overall impression of the efficacy of rufinamide in the SIH paradigm data from all 3 experiments were pooled and analyzed together. ANOVA revealed a significant effect of treatment on the magnitude of the SIH response [F(10, 205)=12.00, p

<0.001]. The SIH response was significantly attenuated by treatment with rufinamide (30, 50, 100, 200 and 300 mg/kg, p.o.) (p< and CDZ (10 mg/kg, p.o.) (FIG. 23). SIH was assessed as the difference in body temperature 15 minutes after stress (the taking of rectal temperature is the stressor) compared to baseline temperature which was determined 60 minutes after subjects were treated p.o. with rufinamide, chlordiazepoxide (CDZ) or vehicle (methylcellulose). Bars represent mean and sem (n=12). *** p<0.001 vs. control.

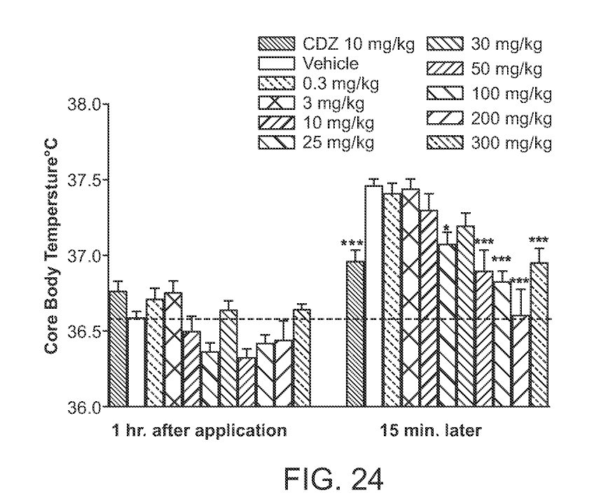

Although ANOVA indicated a significant effect of drug treatment on basal temperature T1 [F(10, 205)=4.39, p<0.001]. no treatment group was significantly different from control animals. In addition, there was a significant effect of drug treatment on T2 [F(10, 205)=10.46, p<0.001]. Post-hoc analysis revealed that animals treated with both rufinamide at the highest doses (25, 50, 100, 200 and 300 mg/kg) or with the positive control CDZ (10 mg/kg, p.o.) had a significant lower body temperature than control animals (FIG. 24). Body temperature was measured 60 minutes after treatment with rufinamide, CDZ, or vehicle (T1) and again 15 minutes later (T2). Bars represent mean and sem. *** denotes p<0.001 versus control.

The present data indicate that rufinamide (>30 mg/kg) elicits a robust reversal of the physiological response (i.e. increase in body temperature) to an acute stressor (taking of rectal temperature) in a manner qualitatively similar to that of the benzodiazepine chlordiazepoxide (10 mg/kg, p.o.). Given that a broad spectrum of anxiolytic agents shares the ability to attenuate SIH, the current data supports the contention that rufinamide may have anxiolytic-like potential. Further, the current data supports the investigation of other effects of rufinamide both in other preclinical tests of anxiety and also in the clinic.

It should be notable that the doses of rufinamide needed to induce robust anxiolytic-like effects (100-300 mg/kg) in the SIH paradigm are much higher than that need to produce anticonvulsant effects in a variety of rodent paradigms.

Furthermore, the anxiolytic doses are also far above that which are sociotropic in the intruder test (3 mg/kg), however for reasons not yet understood anxiolytic-like effects in this test generally occur with higher doses than in ethological based tests.

### Example 9

Rufinamide, was examined in the Vogel Conflict Test in the rat after oral administration to evaluate its potential anxiolytic-like activity.

Rufinamide was dispersed in 0.5% methylcellulose (MC) in distilled water. Vehicle was dispersed in 0.5% MC in distilled water. Clobazam was dispersed in 0.5% MC in distilled water. Methylcellulose (Sigma), batch n° 62K0144, white powder. Distilled water (Laboratoire Aguettant, sterile water for injectable preparation). All substances were stored in a dry, dark, controlled access area and maintained at a controlled ambient temperature (+18° C.). The vehicle suspensions were stored at +4° C.

Substance accountability was carried out according to Porsolt & Partners Pharmacology's standardized internal procedures including records of date of receipt, weight at receipt, dates and amounts of substances used at each preparation (including calculations performed for each concentration and paper print-outs of amounts weighed at each preparation).

Excess quantity of the test substance will be maintained at Porsolt & Partners Pharmacology for 1 year from reception, after which the sponsor will be notified for instructions. Remaining quantities of the commercial substances acquired for the study remain the property of Porsolt & Partners Pharmacology.

The vehicle, test substance and reference substance were administered in a volume of 5 ml/kg body weight.

The doses of rufinamide are expressed in mg/kg of supplied substance.

The dose of clobazam is expressed in mg/kg of base.

Doses were prepared W/V (stock) and then V/V (dilutions), as appropriate.

The suspensions for test and reference substances were freshly prepared on the day of the experiment. Prepared substances were not thereafter subjected to any physico-chemical analyses.

Reference substance was prepared per SOP. The quality of the preparations was verified indirectly from their observed pharmacological effects.

Male Rj: Wistar (Han) rats, 171-205 g body weight range were used. They were supplied by Elevage Janvier, 53940 Le Genest-Saint-Isle, France.

The characteristics of the animals used (age, strain species) were comparable with those described in the scientific literature.

In addition, Porsolt & Partners Pharmacology maintains historical data for tracking biological responses in positive and negative control groups over time for these standard tests and animals.

Animals were delivered to the laboratory at least 5 days before the experiment, during which time they were acclimatized to the laboratory conditions. They were housed in groups of 5 in macrolon cages (41×25×14 cm) on wood litter (Litalabo—SPPS, 95100 Argenteuil, France) with free access to food (Code 113-SAFE, 91360 Epinay-sur-Orge, France) and water until

Raw data were entered into calculation sheets (programmed formulae) previously controlled, checked and protected, using a commercial spreadsheet product (Microsoft Excel®). All data entered were compared with raw data by two people and thus completely verified before data analysis.

Results in grouped data tables are presented as means followed by a ± sign and the standard error of the mean (s.e.m.) on the basis of 15 animals per group and as percent change from control. The statistical tests used are indicated with the method. All statistical calculations were performed using commercial software (Microsoft Excel®, GB Stat version 6.5), and verified test by test according to Porsolt & Partners Pharmacology's standardized internal procedures. All differences were considered statistically significant when the null hypothesis could be rejected at the risk α=0.05 or less.

Rats were deprived of water for 48 hours and were then placed individually into a transparent Plexiglas enclosure (15×32×34 cm) with a floor consisting of stainless steel bars (0.4 cm) spaced 1 cm apart. The back wall of the enclosure was made of opaque Plexiglass thereby concealing the observer from the experimental animal. In the centre of the opposite wall, 5 cm above the floor, a metal water spout protruded into the cage and was connected to one pole of a shock generator (Apelex: Type 011346). The other pole of the shock generator was connected to the metal grid floor.

The rat was left to explore until it found the water spout. Then, every time it drank, it received a slight electric shock (1.7 mA, 1 sec.) 2 seconds after it started lapping. The number of shocks received (punished drinkings) was counted during a 3 minute period.

15 rats were studied per group. The test was performed blind.

rufinamide was evaluated at 25, 50, 100 and 200 mg/kg, administered p.o. 60 minutes before the test, and compared with a vehicle control group.

Clobazam (64 mg/kg), administered under the same experimental conditions, was used as reference substance.

The experiment therefore included 6 groups.

Data were analyzed by comparing treated groups with vehicle control using unpaired Student's t tests.

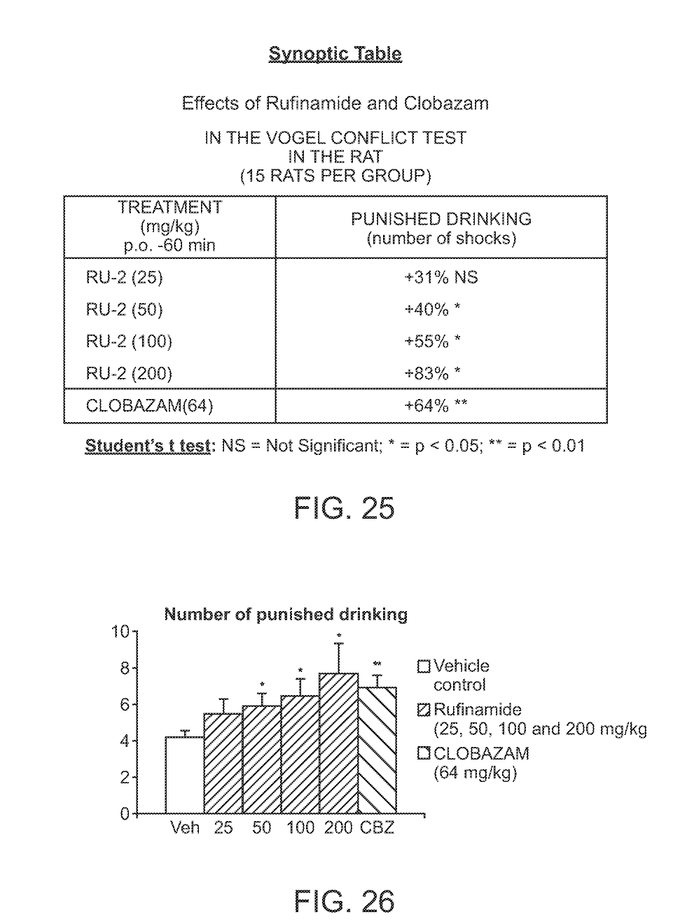

FIG. 26 shows that rufinamide (25, 50, 100 and 200 mg/kg), administered p.o. 60 minutes before the test, dose-dependently increased the number of shocks received (punished drinking), as compared with vehicle control, (+31%, +40%, +55%, +83%, respectively), significantly so from 50 mg/kg (p<0.05). Clobazam (64 mg/kg), administered under the same experimental conditions, significantly increased the number of shocks received (+64%, p<0.01). (See FIG. 25; RU-2 in the figure refers to rufinamide.).

These results suggest anxiolytic-like activity for rufinamide over the dose-range 25-200 mg/kg.

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method of treating a patient diagnosed with post-traumatic stress disorder, comprising administering to the patient a therapeutically effective amount of rufinamide (1-(2,6-difluorobenzyl)-1H-1,2,3-triazole-4-carboxamide).

2. The method of claim 1, wherein the rufinamide reduces kindling in the patient.

3. The method of claim 1, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

4. The method of claim 3, wherein the at least one other agent is a SSRI selected from paroxetine, sertraline, citalopram, escitalopram, and fluoxetine.

5. The method of claim 3, wherein the at least one other agent is a SNRI selected from duloxetine, mirtazapine, and venlafaxine.

6. The method of claim 3, wherein the at least one other agent is a NRI selected from bupropion and atomoxetine.

7. The method of claim 1, wherein the rufinamide reduces at least one sodium channel activity in the patient.

8. The method of claim 1, wherein the rufinamide reduces at least one of the frequency and intensity of at least one sign of the post-traumatic stress disorder in the patient.

9. The method of claim 1, wherein the rufinamide reduces at least one of the frequency and intensity of at least one symptom of the post-traumatic stress disorder in the patient.

10. The method of claim 1, wherein the rufinamide reduces at least one of the frequency and intensity of at least one symptom cluster of the post-traumatic stress disorder in the patient, wherein the symptom cluster is selected from re-experiencing/intrusion, avoidance/numbing, and hyperarousal.

11. The method of claim 23, wherein the re-experiencing/intrusion comprises at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders.

12. The method of claim 1, wherein the patient is a child or an adolescent.

13. The method of claim 12, wherein the rufinamide reduces at least one of the frequency and intensity of at least one sign or symptom of the post-traumatic stress disorder in the patient, wherein the sign or symptom is selected from disorganized or agitated behavior, repetitive play that expresses aspects of the trauma, frightening dreams which lack recognizable content, and trauma-specific reenactment.

14. The method of claim 1, wherein the rufinamide reduces the incidence of at least one disorder comorbid with post-traumatic stress disorder selected from drug abuse, alcohol abuse, and depression in the patient.

15. A method of treating kindling in a patient, comprising administering to the patient a therapeutically effective amount of rufinamide.

16. The method of claim 15, wherein the rufinamide reduces the incidence of at least one of epilepsy and post-traumatic stress disorder in the patient.

17. The method of claim 16, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

18. The method of claim 16, wherein the rufinamide reduces at least one of the frequency and intensity of at least one sign of the post-traumatic stress disorder in the patient.

19. The method of claim 16, wherein the rufinamide reduces at least one of the frequency and intensity of at least one symptom of the post-traumatic stress disorder in the patient.

20. A method of treating post-traumatic stress disorder in a patient comprising:
diagnosing the patient with post-traumatic stress disorder;
administering to the patient a therapeutically effective amount of rufinamide;
assessing at least one of sign, symptom, and symptom cluster of post-traumatic stress disorder; and
determining that the post-traumatic stress syndrome is improved if the rufinamide reduces at least one of sign, symptom, and symptom cluster of post-traumatic stress disorder.

21. The method of claim 20, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

22. The method of claim 20, wherein the rufinamide reduces at least one of the frequency and intensity of at least one sign of the post-traumatic stress disorder in the patient.

23. A method of diagnosing post-traumatic stress disorder in a patient comprising:
administering to the patient a therapeutically effective amount of rufinamide and
assessing at least one of sign, symptom, or symptom cluster of post-traumatic stress disorder; and
diagnosing post-traumatic stress disorder in the patient if the rufinamide reduces at least one of sign, symptom, and symptom cluster of post-traumatic stress disorder.

24. The method of claim 23, wherein the patient is a child, adolescent, or adult.
